SEQUENCE LISTING (288 sequences)

SEQ ID NO: 1 - DNA, 19 bp, Artificial
  Probe for Enterobacter cloacae
acgtcaattgctgcggtta

SEQ ID NO: 2 - DNA, 19 bp, Artificial
  Probe for Staphylococcus aureus
agcaagcttctcgtccgtt

SEQ ID NO: 3-18 - skipped (16 entries)

SEQ ID NO: 19 - DNA, 19 bp, Artificial
  Probe for Escherichia coli
agcaagcccttctgctgtt

SEQ ID NO: 20 - DNA, 19 bp, Artificial
  Probe for Escherichia coli
ggcagtctctctttgagtt

SEQ ID NO: 21 - DNA, 19 bp, Artificial
  Probe for Escherichia coli
tcagactacgcacgacttt

SEQ ID NO: 22 - DNA, 19 bp, Artificial
  Probe for Enterococcus faecalis
gccatgcggcataaactgt

SEQ ID NO: 23 - DNA, 19 bp, Artificial
  Probe for Enterococcus faecalis
cgaaagcgcctttcactct

SEQ ID NO: 24 - DNA, 19 bp, Artificial
  Probe for Enterococcus faecalis
agataccgtcaggggacgt

SEQ ID NO: 25 - DNA, 19 bp, Artificial
  Probe for Klebsiella pneumoniae
ttcctccccactgaaagtg

SEQ ID NO: 26 - DNA, 19 bp, Artificial
  Probe for Klebsiella pneumoniae
ggtaacgtcaatcgccaag

SEQ ID NO: 27 - DNA, 19 bp, Artificial
  Probe for Klebsiella pneumoniae
tgcgggtaacgtcaatcgc

SEQ ID NO: 28 - DNA, 19 bp, Artificial
  Probe for Staphylococcus epidermidis (excludes Staphylococcusaureus)
tcactattgaaccatgcgg

SEQ ID NO: 29 - DNA, 19 bp, Artificial
  Probe for Staphylococcus aureus (excludes Staphylococcus epidermidis)
ccgtcaagatgtgcacagt

SEQ ID NO: 30 - DNA, 19 bp, Artificial
  Probe for Pseudomonas aeruginosa
gatcccccactttctccct

SEQ ID NO: 31 - DNA, 19 bp, Artificial
  Probe for Streptococcus pneumoniae
tgtcatgcaacatccactc

SEQ ID NO: 32 - DNA, 19 bp, Artificial
  Probe for Streptococcus pneumoniae
cgtgaacgtagtgatggtc

SEQ ID NO: 33 - DNA, 19 bp, Artificial
  Probe for Propionibacterium acnes
tttcaaagccgccaacccc

SEQ ID NO: 34 - DNA, 19 bp, Artificial
  Probe for Pseudomonas aeruginosa
gcggtattagcgcccgttt

SEQ ID NO: 35 - DNA, 19 bp, Artificial
  Probe for Pseudomonas aeruginosa
actttctccctcaggacgt

SEQ ID NO: 36 - DNA, 19 bp, Artificial
  Probe for Propionibacterium acnes
cccacaaaagcagggcctt

SEQ ID NO: 37 - DNA, 19 bp, Artificial
  Probe for Streptococcus pneumoniae
ctggtagtgatgcaagtgc

SEQ ID NO: 38 - DNA, 19 bp, Artificial
  Probe for Streptococcus pneumoniae
tctggtagtgatgcaagtg

SEQ ID NO: 39 - DNA, 19 bp, Artificial
  Probe for Streptococcus pneumoniae
catctggtagtgatgcaag

SEQ ID NO: 40 - DNA, 19 bp, Artificial
  Probe for Streptococcus pyogenes
taaattactaacatgcgtt

SEQ ID NO: 41 - DNA, 19 bp, Artificial
  Probe for Streptococcus pyogenes
aattactaacatgcgttag

SEQ ID NO: 42 - DNA, 19 bp, Artificial
  Probe for Streptococcus pyogenic consensus
aattgcaccttttaaatga

SEQ ID NO: 43 - DNA, 19 bp, Artificial
  Probe for Streptococcus pyogenic consensus
taacttcagacttaaagaa

SEQ ID NO: 44 - DNA, 19 bp, Artificial
  Probe for Streptococcus salivarius
aataaatgacatgtgtcat

SEQ ID NO: 45 - DNA, 19 bp, Artificial
  Probe for Streptococcus salivarius
aaataaatgacatgtgtca

SEQ ID NO: 46 - DNA, 19 bp, Artificial
  Probe for Streptococcus salivarius consensus
ctatctctagaaatagcat

SEQ ID NO: 47 - DNA, 19 bp, Artificial
  Probe for Streptococcus salivarius consensus
ctatctctagaaatagcat

SEQ ID NO: 48 - DNA, 19 bp, Artificial
  Probe for Streptococcus sanguis
atgcaataatcaattttat

SEQ ID NO: 49 - DNA, 19 bp, Artificial
  Probe for Streptococcus sanguis
gcatctttcaattaattat

SEQ ID NO: 50 - DNA, 19 bp, Artificial
  Probe for Enterobacter cloacae
ggtcttgcgacgttatgcg

SEQ ID NO: 51 - DNA, 19 bp, Artificial
  Probe for Staphylococcus consensus
cacttttgaaccatgcggt

SEQ ID NO: 52 - DNA, 19 bp, Artificial
  Probe for Staphylococcus consensus
caccccaatcatttgtccc

SEQ ID NO: 53 - DNA, 19 bp, Artificial
  Probe for Bacteroides fragilis
gaacgcatccccatccttt

SEQ ID NO: 54 - DNA, 19 bp, Artificial
  Probe for Enterobacter agglomerans
ttcagtagtacgggaatgc

SEQ ID NO: 55 - DNA, 19 bp, Artificial
  Probe for Streptococcus dysgalactiae
ttgcaccttttaaatgaaa

SEQ ID NO: 56 - DNA, 19 bp, Artificial
  Probe for Streptococcus equinus
cttctttcaagcatctaac

SEQ ID NO: 57 - DNA, 19 bp, Artificial
  Probe for Streptococcus intermedius
gtatgaactttccattctc

SEQ ID NO: 58 - DNA, 19 bp, Artificial
  Probe for Streptococcus mitis
tctacttgcatgtattagg

SEQ ID NO: 59 - DNA, 19 bp, Artificial
  Probe for Streptococcus mitis
aaaactctatctctagagc

SEQ ID NO: 60 - DNA, 19 bp, Artificial
  Probe for Streptococcus mitis consensus
accttttaagtaaatgtca

SEQ ID NO: 61 - DNA, 19 bp, Artificial
  Probe for Streptococcus mitis consensus
accttttaagtaaatgtca

SEQ ID NO: 62 - DNA, 19 bp, Artificial
  Probe for Streptococcus mitis consensus
aattgcaccttttaagtaa

SEQ ID NO: 63 - DNA, 19 bp, Artificial
  Probe for Streptococcus mitis consensus
aattgcaccttttaagtaa

SEQ ID NO: 64 - DNA, 19 bp, Artificial
  Probe for Streptococcus mutans
cttgcatctttcaatcaat

SEQ ID NO: 65 - DNA, 19 bp, Artificial
  Probe for Streptococcus mutans
attatcatgcaataattaa

SEQ ID NO: 66 - DNA, 19 bp, Artificial
  Probe for Streptococcus mutans consensus
tttaacttcagacttacta

SEQ ID NO: 67 - DNA, 19 bp, Artificial
  Probe for Streptococcus mutans consensus
atctttcaatcaattaaca

SEQ ID NO: 68 - DNA, 19 bp, Artificial
  Probe for Streptococcus pneumonia
actagctaatacaacgtag

SEQ ID NO: 69 - DNA, 19 bp, Artificial
  Probe for Streptococcus pneumonia
actagctaatacaacgtag

SEQ ID NO: 70 - DNA, 19 bp, Artificial
  Probe for Streptococcus pneumonia consensus
aaagcctactatggttaag

SEQ ID NO: 71 - DNA, 19 bp, Artificial
  Probe for Streptococcus pneumoniae
gtcatgcaacatccactct

SEQ ID NO: 72 - DNA, 19 bp, Artificial
  Probe for Streptococcus pneumoniae
tgtcatgcaacatccactc

SEQ ID NO: 73 - DNA, 19 bp, Artificial
  Probe for Staphylococcus kloosii
tctataagtgatagcaagg

SEQ ID NO: 74 - DNA, 19 bp, Artificial
  Probe for Staphylococcus lugdunensis
agagttttacgatcctaag

SEQ ID NO: 75 - DNA, 19 bp, Artificial
  Probe for Staphylococcus saprophyticu
cacagttacttacacattt

SEQ ID NO: 76 - DNA, 19 bp, Artificial
  Probe for Staphylococcus saprophyticu
agttacttacacatttgtt

SEQ ID NO: 77 - DNA, 19 bp, Artificial
  Probe for Staphylococcus sciuri
ttcagttactaacaaattt

SEQ ID NO: 78 - DNA, 19 bp, Artificial
  Probe for Staphylococcus simulans
gcatagttacttacatcct

SEQ ID NO: 79 - DNA, 19 bp, Artificial
  Probe for Staphylococcus warneri
ttcaatatgttatccggta

SEQ ID NO: 80 - DNA, 19 bp, Artificial
  Probe for Staphylococcus xylosus
tctaaatgttatccggtat

SEQ ID NO: 81 - DNA, 19 bp, Artificial
  Probe for Stomatococcus mucilaginosus
cagagttaaaggtaggtta

SEQ ID NO: 82 - DNA, 19 bp, Artificial
  Probe for Streptococcus agalactiae
taacatgtgttaattactc

SEQ ID NO: 83 - DNA, 19 bp, Artificial
  Probe for Streptococcus agalactiae
atcagtctagtgtaaacac

SEQ ID NO: 84 - DNA, 19 bp, Artificial
  Probe for Streptococcus agalactiae
ctcatcagtctagtgtaaa

SEQ ID NO: 85 - DNA, 19 bp, Artificial
  Probe for Streptococcus anginosus consensus
acagtatgaactttccatt

SEQ ID NO: 86 - DNA, 19 bp, Artificial
  Probe for Streptococcus anginosus consensus
tttcacttcagacttatct

SEQ ID NO: 87 - DNA, 19 bp, Artificial
  Probe for Streptococcus bovis consensus
tgtgttaaatgctgttatg

SEQ ID NO: 88 - DNA, 19 bp, Artificial
  Probe for Streptococcus bovis consensus
cttcagacttattaaaccg

SEQ ID NO: 89 - DNA, 19 bp, Artificial
  Probe for Streptococcus bovis consensus
cttcctatctctaggaata

SEQ ID NO: 90 - DNA, 19 bp, Artificial
  Probe for Streptococcus canis
gttcttaacatgtgttaag

SEQ ID NO: 91 - DNA, 19 bp, Artificial
  Probe for Citrobacter freundi
tatcgaattaaaccacatg

SEQ ID NO: 92 - DNA, 19 bp, Artificial
  Probe for Burkholderia cepacia
tagaaccaaggatttcttt

SEQ ID NO: 93 - DNA, 19 bp, Artificial
  Probe for Bacteroides distasonis
cttataaaagaggtttacg

SEQ ID NO: 94 - DNA, 19 bp, Artificial
  Probe for Actinomyces meyeri
cagtgaatatccagtatta

SEQ ID NO: 95 - DNA, 19 bp, Artificial
  Probe for Staphylococcus epidermidis
cagcaaaaccgtctttcac

SEQ ID NO: 96 - DNA, 19 bp, Artificial
  Probe for Serratia marcescen
tcaattgatgagcgtatta

SEQ ID NO: 97 - DNA, 19 bp, Artificial
  Probe for Pseudomonas aeruginosa
gcggtattagcgcccgttt

SEQ ID NO: 98 - DNA, 19 bp, Artificial
  Probe for Proteus mirabilis
gtaacgtcaattgataagg

SEQ ID NO: 99 - DNA, 19 bp, Artificial
  Probe for PeptoStreptococcus consensus
tagcagttttaaatgctta

SEQ ID NO: 100 - DNA, 19 bp, Artificial
  Probe for Legionella pneumophila
ttaatcagctcttaaccta

SEQ ID NO: 101 - DNA, 19 bp, Artificial
  Probe for Klebsiella oxytoca
ataaggttattaacctcac

SEQ ID NO: 102 - DNA, 19 bp, Artificial
  Probe for Gemella haemolysans
aacttttaaacatcaacca

SEQ ID NO: 103 - DNA, 19 bp, Artificial
  Probe for Staphylococcus epidermidis
acatcagcgtcagatacag

SEQ ID NO: 104 - DNA, 19 bp, Artificial
  Probe for Staphylococcus epidermidis
tcgcacatcagcgtcagat

SEQ ID NO: 105 - DNA, 19 bp, Artificial
  Probe for Staphylococcus epidermidis
ctattgaaccatgcggttc

SEQ ID NO: 106 - DNA, 19 bp, Artificial
  Probe for Staphylococcus haemolyticus
tagttacttacacgtatgt

SEQ ID NO: 107 - DNA, 19 bp, Artificial
  Probe for Staphylococcus hominis
tctataagtgatagcagag

SEQ ID NO: 108 - DNA, 19 bp, Artificial
  Probe for Staphylococcus hominis
tctataagtgatagcagag

SEQ ID NO: 109 - DNA, 19 bp, Artificial
  Probe for Enterobacter agglomerans
cgtaagggcatgatgactt

SEQ ID NO: 110 - DNA, 19 bp, Artificial
  Probe for Clostridium ramosum
ctttgaggcacttttaata

SEQ ID NO: 111 - DNA, 19 bp, Artificial
  Probe for Citrobacter freundi
tatcgaattaaaccacatg

SEQ ID NO: 112 - DNA, 19 bp, Artificial
  Probe for Bacteroides vulgatus
tccttattcataaagtaca

SEQ ID NO: 113 - DNA, 19 bp, Artificial
  Probe for Bacteroides consensus
tttccacataattcagttg

SEQ ID NO: 114 - DNA, 19 bp, Artificial
  Probe for Actinomyces meyeri
ccagtgaatatccagtatt

SEQ ID NO: 115 - DNA, 19 bp, Artificial
  Probe for Staphylococcus cohnii
taaatgttatccggcatta

SEQ ID NO: 116 - DNA, 19 bp, Artificial
  Probe for Staphylococcus epidermidis
catgcggttcaatatatta

SEQ ID NO: 117 - DNA, 19 bp, Artificial
  Probe for Pseudomonas aeruginosa
tgttccttcctatatctac

SEQ ID NO: 118 - DNA, 19 bp, Artificial
  Probe for Propionibacterium consensus
ttaccttgttacgacttag

SEQ ID NO: 119 - DNA, 19 bp, Artificial
  Probe for PeptoStreptococcus consensus
tgctggtaactaaagatag

SEQ ID NO: 120 - DNA, 19 bp, Artificial
  Probe for Legionella pneumophila
aatcagctcttaacctatc

SEQ ID NO: 121 - DNA, 19 bp, Artificial
  Probe for Klebsiella pneumoniae
ggtaacgtcaatgaataag

SEQ ID NO: 122 - DNA, 19 bp, Artificial
  Probe for Gemella haemolysans
tgtatagttactacacaat

SEQ ID NO: 123 - DNA, 19 bp, Artificial
  Probe for Eubacterium limosum
aaaaccataatataaggct

SEQ ID NO: 124 - DNA, 19 bp, Artificial
  Probe for Enterococcus faecalis
gccactcctctttccaatt

SEQ ID NO: 125 - DNA, 19 bp, Artificial
  Probe for Enterobacter agglomerans
ggggatttcacatcgactt

SEQ ID NO: 126 - DNA, 19 bp, Artificial
  Probe for Clostridium tertium
gctcctttaattacttctt

SEQ ID NO: 127 - DNA, 19 bp, Artificial
  Probe for Escherichia coli
cgctgaaagtacgtggctt

SEQ ID NO: 128 - DNA, 19 bp, Artificial
  Probe for Enterococcus faecali
ctttaagagatttgcatga

SEQ ID NO: 129 - DNA, 19 bp, Artificial
  Probe for Enterobacter agglomerans
cctttgagttcccaccatt

SEQ ID NO: 130 - DNA, 19 bp, Artificial
  Probe for Clostridium perfringens
tcaacattatgcggtatta

SEQ ID NO: 131 - DNA, 19 bp, Artificial
  Probe for Citrobacter freundi
ttcctctagtttatcactg

SEQ ID NO: 132 - DNA, 19 bp, Artificial
  Probe for Bacteroides ureolyticus
attctttcctgataaaagg

SEQ ID NO: 133 - DNA, 19 bp, Artificial
  Probe for Bacteroides consensus
cacataattcagttgcaat

SEQ ID NO: 134 - DNA, 19 bp, Artificial
  Probe for Acinetobacter junii
aagagtattagtctcagta

SEQ ID NO: 135 - DNA, 19 bp, Artificial
  Probe for Staphylococcus aureus
aagcaagcttctcgtccgt

SEQ ID NO: 136 - DNA, 19 bp, Artificial
  Probe for Staphylococcus epidermidis
tgcggttcaatatattatc

SEQ ID NO: 137 - DNA, 19 bp, Artificial
  Probe for Pseudomonas aeruginosa
tcctatatctacgcatttc

SEQ ID NO: 138 - DNA, 19 bp, Artificial
  Probe for Propionibacterium consensus
gttacgacttagtcctaat

SEQ ID NO: 139 - DNA, 19 bp, Artificial
  Probe for PeptoStreptococcus anaerobi
actttgatatatctacgat

SEQ ID NO: 140 - DNA, 19 bp, Artificial
  Probe for Lactobacillus consensus
aagcaccattcattattaa

SEQ ID NO: 141 - DNA, 19 bp, Artificial
  Probe for Haemophilus paraphrophilus
tttcatctctcgattctac

SEQ ID NO: 142 - DNA, 19 bp, Artificial
  Probe for Fusobacterium varium
atagctttcataaccaaat

SEQ ID NO: 143 - DNA, 19 bp, Artificial
  Probe for Escherichia coli
aacgcacatcatgcgtctt

SEQ ID NO: 144 - DNA, 19 bp, Artificial
  Probe for Enterococcus faecalis
cgaaagcgcctttcactct

SEQ ID NO: 145 - DNA, 19 bp, Artificial
  Probe for Haemophilus parainfluenzae
agtctattaaactaaatgc

SEQ ID NO: 146 - DNA, 19 bp, Artificial
  Probe for Fusobacterium nucleatum
tagctttcataattctaag

SEQ ID NO: 147 - DNA, 19 bp, Artificial
  Probe for Escherichia coli
aaccgactccatgaagtcg

SEQ ID NO: 148 - DNA, 19 bp, Artificial
  Probe for Enterococcus faecali
gagaagctttaagagattt

SEQ ID NO: 149 - DNA, 19 bp, Artificial
  Probe for Enterobacter aerogenes
caaggttattaaccttaac

SEQ ID NO: 150 - DNA, 19 bp, Artificial
  Probe for Clostridium perfringens
acattatgcggtattaatc

SEQ ID NO: 151 - DNA, 19 bp, Artificial
  Probe for Citrobacter freundi
cttcctctagtttatcact

SEQ ID NO: 152 - DNA, 19 bp, Artificial
  Probe for Bacteroides thetaiotaomicro
ccgaaattctttaataata

SEQ ID NO: 153 - DNA, 19 bp, Artificial
  Probe for Bacteroides consensus
cagtatcaactgcaatttt

SEQ ID NO: 154 - DNA, 19 bp, Artificial
  Probe for Acinetobacter consensus
gagatgatatccggtatta

SEQ ID NO: 155 - DNA, 19 bp, Artificial
  Probe for Escherichia coli
agcaagcttctcgtccgtt

SEQ ID NO: 156 - DNA, 19 bp, Artificial
  Probe for Staphylococcus epidermidis
atgcggttcaatatattat

SEQ ID NO: 157 - DNA, 19 bp, Artificial
  Probe for Pseudomonas aeruginosa
gttccttcctatatctacg

SEQ ID NO: 158 - DNA, 19 bp, Artificial
  Probe for Propionibacterium acnes
agttagccggtgcttcttt

SEQ ID NO: 159 - DNA, 19 bp, Artificial
  Probe for PeptoStreptococcus anaerobi
catgtattagtaaactttt

SEQ ID NO: 160 - DNA, 19 bp, Artificial
  Probe for Lactobacillus consensus
ggatcaaactctcatttta

SEQ ID NO: 161 - DNA, 19 bp, Artificial
  Probe for Haemophilus paraphrophilus
attaacgtcaatttgttgt

SEQ ID NO: 162 - DNA, 19 bp, Artificial
  Probe for Fusobacterium nucleatum
taattctaagatgccttaa

SEQ ID NO: 163 - DNA, 19 bp, Artificial
  Probe for Pasteurella multocida
gctatctatttaacaacat

SEQ ID NO: 164 - DNA, 19 bp, Artificial
  Probe for Klebsiella pneumoniae
cgggtaacgtcaatcgatg

SEQ ID NO: 165 - DNA, 19 bp, Artificial
  Probe for Haemophilus parainfluenzae
ctagtctattaaactaaat

SEQ ID NO: 166 - DNA, 19 bp, Artificial
  Probe for Fusobacterium necrophorum
aaaaccataatatccggta

SEQ ID NO: 167 - DNA, 19 bp, Artificial
  Probe for Escherichia coli
aattcgatttgagttttaa

SEQ ID NO: 168 - DNA, 19 bp, Artificial
  Probe for Enterococcus consensus
gatgaacattctactctca

SEQ ID NO: 169 - DNA, 19 bp, Artificial
  Probe for Enterobacter aerogenes
aaggttattaaccttaacg

SEQ ID NO: 170 - DNA, 19 bp, Artificial
  Probe for Clostridium paraputrificum
tttaattgctacttcatgc

SEQ ID NO: 171 - DNA, 19 bp, Artificial
  Probe for Citrobacter amalonaticus
aatggctaaggttattaac

SEQ ID NO: 172 - DNA, 19 bp, Artificial
  Probe for Bacteroides thetaiotaomicro
ccgaaattctttaataata

SEQ ID NO: 173 - DNA, 19 bp, Artificial
  Probe for Bacteroides caccae
accgaatttctttaatata

SEQ ID NO: 174 - DNA, 19 bp, Artificial
  Probe for Acinetobacter consensus
gagatgatatccggtatta

SEQ ID NO: 175 - DNA, 19 bp, Artificial
  Probe for Staphylococcus aureus
cgtggctttctgattaggt

SEQ ID NO: 176 - DNA, 19 bp, Artificial
  Probe for Salmonella enteritidi
gtatataatcctgtttgct

SEQ ID NO: 177 - DNA, 19 bp, Artificial
  Probe for Pseudomonas aeruginosa
cttcctatatctacgcatt

SEQ ID NO: 178 - DNA, 19 bp, Artificial
  Probe for Propionibacterium acnes
cccacaaaagcagggcctt

SEQ ID NO: 179 - DNA, 19 bp, Artificial
  Probe for Pasteurella multocida
taattaacgtcaatgatgc

SEQ ID NO: 180 - DNA, 19 bp, Artificial
  Probe for Lactobacillus consensus
ctaaggttgtcaaaagatg

SEQ ID NO: 181 - DNA, 19 bp, Artificial
  Probe for Providencia stuartii
cgttgatggtattaacatc

SEQ ID NO: 182 - DNA, 19 bp, Artificial
  Probe for Propionibacterium acne
caattcctttgagttttag

SEQ ID NO: 183 - DNA, 19 bp, Artificial
  Probe for Neisseria flavescens
ccaactaactaatcagata

SEQ ID NO: 184 - DNA, 19 bp, Artificial
  Probe for Klebsiella pneumoniae
tgcgggtaacgtcaatcgc

SEQ ID NO: 185 - DNA, 19 bp, Artificial
  Probe for Haemophilus influenzae
ttaacgtcaatttgatgta

SEQ ID NO: 186 - DNA, 19 bp, Artificial
  Probe for Fusobacterium necrophorum
catctagctttcatgattc

SEQ ID NO: 187 - DNA, 19 bp, Artificial
  Probe for Escherichia coli
tacttcttttggacgaata

SEQ ID NO: 188 - DNA, 19 bp, Artificial
  Probe for Enterococcus consensus
gagaagctttaagagatta

SEQ ID NO: 189 - DNA, 19 bp, Artificial
  Probe for Eikenella corrodens
actagctaatcagttatcg

SEQ ID NO: 190 - DNA, 19 bp, Artificial
  Probe for Clostridium innocuum
gctcagtcaatttaaattc

SEQ ID NO: 191 - DNA, 19 bp, Artificial
  Probe for Citrobacter amalonaticus
aaggttattaaccttaacc

SEQ ID NO: 192 - DNA, 19 bp, Artificial
  Probe for Bacteroides stercoris
aattctttaataatcatcc

SEQ ID NO: 193 - DNA, 19 bp, Artificial
  Probe for Bacteroides caccae
tatgctatcggatattaat

SEQ ID NO: 194 - DNA, 19 bp, Artificial
  Probe for Acinetobacter consensus
agatgatatccggtattag

SEQ ID NO: 195 - DNA, 19 bp, Artificial
  Probe for Staphylococcus aureus
tagctaatgcagcgcggat

SEQ ID NO: 196 - DNA, 19 bp, Artificial
  Probe for Salmonella consensus
attctcatctctgaaaact

SEQ ID NO: 197 - DNA, 19 bp, Artificial
  Probe for Providencia stuartii
caatcgttgatggtattaa

SEQ ID NO: 198 - DNA, 19 bp, Artificial
  Probe for Propionibacterium acnes
ccccaaccgccgaaacttt

SEQ ID NO: 199 - DNA, 19 bp, Artificial
  Probe for Staphylococcus aureus
gcaagaccgtctttcactt

SEQ ID NO: 200 - DNA, 19 bp, Artificial
  Probe for Salmonella consensus
tgatcaaactcttcaattt

SEQ ID NO: 201 - DNA, 19 bp, Artificial
  Probe for Proteus vulgaris
gctaagagtattaatctta

SEQ ID NO: 202 - DNA, 19 bp, Artificial
  Probe for Aerococcus viridans
aggaggacataaggtatta

SEQ ID NO: 203 - DNA, 19 bp, Artificial
  Probe for Neisseria flavescens
accaactaactaatcagat

SEQ ID NO: 204 - skipped

SEQ ID NO: 205 - DNA, 19 bp, Artificial
  Probe for Haemophilus influenzae
taacgtcaatttgatgtac

SEQ ID NO: 206 - DNA, 19 bp, Artificial
  Probe for Fusobacterium mortiferum
tatagctttcatatgaatt

SEQ ID NO: 207 - DNA, 19 bp, Artificial
  Probe for Escherichia coli
gatctctactggaattcta

SEQ ID NO: 208 - DNA, 19 bp, Artificial
  Probe for Enterobacter cloacae
acgtcaattgctgcggtta

SEQ ID NO: 209 - DNA, 19 bp, Artificial
  Probe for Eikenella corrodens
caactagctaatcagttat

SEQ ID NO: 210 - DNA, 19 bp, Artificial
  Probe for Clostridium innocuum
gctcagtcaatttaaattc

SEQ ID NO: 211 - DNA, 19 bp, Artificial
  Probe for Chryseomonas luteola
caacgtattaggttacaac

SEQ ID NO: 212 - DNA, 19 bp, Artificial
  Probe for Bacteroides ovatus
atatcatgcgatattcgta

SEQ ID NO: 213 - DNA, 19 bp, Artificial
  Probe for Alcaligenes faecalis
cactctttcgagtagttat

SEQ ID NO: 214 - DNA, 19 bp, Artificial
  Probe for Acinetobacter consensus
agatgatatccggtattag

SEQ ID NO: 215 - DNA, 19 bp, Artificial
  Probe for Staphylococcus aureus
cacttttgaaccatgcggt

SEQ ID NO: 216 - DNA, 19 bp, Artificial
  Probe for Salmonella consensus
ttctcatctctgaaaactt

SEQ ID NO: 217 - DNA, 19 bp, Artificial
  Probe for Aeromonas hydrophila
acagttgatacgtattagg

SEQ ID NO: 218 - DNA, 19 bp, Artificial
  Probe for Acinetobacter consensus
ttttgagattagcatccta

SEQ ID NO: 219 - DNA, 19 bp, Artificial
  Probe for Staphylococcus aureus
tcgctgccctttgtattgt

SEQ ID NO: 220 - DNA, 19 bp, Artificial
  Probe for Staphylococcus aureus
tatctctagagttgtcaaa

SEQ ID NO: 221 - DNA, 19 bp, Artificial
  Probe for Proteus vulgaris
taagagtattaatcttaac

SEQ ID NO: 222 - DNA, 19 bp, Artificial
  Probe for PeptoStreptococcus prevotii
agagatcatttaagcttca

SEQ ID NO: 223 - DNA, 19 bp, Artificial
  Probe for Morganella morganii
aaggttattaaccttgaca

SEQ ID NO: 224 - DNA, 19 bp, Artificial
  Probe for Klebsiella pneumonia
gatgaggttattaacctca

SEQ ID NO: 225 - DNA, 19 bp, Artificial
  Probe for Haemophilus aphrophilus
tacaagtacttacctgtta

SEQ ID NO: 226 - DNA, 19 bp, Artificial
  Probe for Fusobacterium mortiferum
atagctttcatatgaattt

SEQ ID NO: 227 - DNA, 19 bp, Artificial
  Probe for Escherichia coli
atgagcaaagtattagact

SEQ ID NO: 228 - DNA, 19 bp, Artificial
  Probe for Enterobacter cloacae
ggtcttgcgactttatgcg

SEQ ID NO: 229 - DNA, 19 bp, Artificial
  Probe for Corynebacterium consensus
taaagtatggtgtcctatc

SEQ ID NO: 230 - DNA, 19 bp, Artificial
  Probe for Clostridium consensus
atgcgatactctgatatta

SEQ ID NO: 231 - DNA, 19 bp, Artificial
  Probe for Capnocytophaga ochracea
gctttaatagttgtgtgat

SEQ ID NO: 232 - DNA, 19 bp, Artificial
  Probe for Bacteroides fragilis
ggaacgcatccccatcctt

SEQ ID NO: 233 - DNA, 19 bp, Artificial
  Probe for Aeromonas hydrophila
tcacagttgatacgtatta

SEQ ID NO: 234 - DNA, 19 bp, Artificial
  Probe for Acinetobacter consensus
ctctggaaagttcttacta

SEQ ID NO: 235 - DNA, 19 bp, Artificial
  Probe for Capnocytophaga consensus
tcaattaattgttagtaat

SEQ ID NO: 236 - DNA, 19 bp, Artificial
  Probe for Bacteroides fragili
tgttagtaactaaagataa

SEQ ID NO: 237 - DNA, 19 bp, Artificial
  Probe for Aerococcus viridans
gtggctttctgataagata

SEQ ID NO: 238 - DNA, 19 bp, Artificial
  Probe for Acinetobacter consensus
tattaaccaaagtagcctc

SEQ ID NO: 239 - DNA, 19 bp, Artificial
  Probe for Shigella dysenteria
aaaggtattaactttactc

SEQ ID NO: 240 - DNA, 19 bp, Artificial
  Probe for Staphylococcus aureus
tatctaatcctgtttgatc

SEQ ID NO: 241 - DNA, 19 bp, Artificial
  Probe for Proteus penneri
acgtcaattgataaaggta

SEQ ID NO: 242 - DNA, 19 bp, Artificial
  Probe for PeptoStreptococcus prevotii
gaagagatcatttaagctt

SEQ ID NO: 243 - DNA, 19 bp, Artificial
  Probe for Morganella morganii
caaggttattaaccttgac

SEQ ID NO: 244 - DNA, 19 bp, Artificial
  Probe for Klebsiella pneumonia
taattccgattaacgctta

SEQ ID NO: 245 - DNA, 19 bp, Artificial
  Probe for Haemophilus aphrophilus
gtacaagtacttacctgtt

SEQ ID NO: 246 - DNA, 19 bp, Artificial
  Probe for Fusobacterium consensus
attcctttgagtttcatac

SEQ ID NO: 247 - DNA, 19 bp, Artificial
  Probe for Escherichia coli
catgatcaaactctcaatt

SEQ ID NO: 248 - DNA, 19 bp, Artificial
  Probe for Enterobacter cloacae
ggtcttgcgactttatgcg

SEQ ID NO: 249 - DNA, 19 bp, Artificial
  Probe for Corynebacterium consensus
catcgaattaatccacatg

SEQ ID NO: 250 - DNA, 19 bp, Artificial
  Probe for Clostridium consensus
tttcacatctgacttaaat

SEQ ID NO: 251 - DNA, 19 bp, Artificial
  Probe for Capnocytophaga consensus
tctccaactagctaataga

SEQ ID NO: 252 - DNA, 19 bp, Artificial
  Probe for Bacteroides fragilis
catcctttaccggaatcct

SEQ ID NO: 253 - DNA, 19 bp, Artificial
  Probe for CNS (Coagulase Negative Staphylococci) consensus
ttaccaactagctaatacg

SEQ ID NO: 254 - DNA, 19 bp, Artificial
  Probe for Clostridium bifermentans
ccgtattagtatacctttc

SEQ ID NO: 255 - DNA, 19 bp, Artificial
  Probe for Burkholderia cepacia
ctgtattagaaccaaggat

SEQ ID NO: 256 - DNA, 19 bp, Artificial
  Probe for Bacteroides fragili
ctgcactttattcttatat

SEQ ID NO: 257 - DNA, 19 bp, Artificial
  Probe for Aerococcus viridans
gcttataggtagattcctt

SEQ ID NO: 258 - DNA, 19 bp, Artificial
  Probe for Acinetobacter calcoaceticus
ctgaaggtattaacttcag

SEQ ID NO: 259 - DNA, 19 bp, Artificial
  Probe for Shigella dysenteria
gtatctctacaaggttctg

SEQ ID NO: 260 - DNA, 19 bp, Artificial
  Probe for Staphylococcus aureus
cacagttacttacacatat

SEQ ID NO: 261 - DNA, 19 bp, Artificial
  Probe for Proteus penneri
taacgtcaattgataaagg

SEQ ID NO: 262 - DNA, 19 bp, Artificial
  Probe for PeptoStreptococcus micros
agaatttccacaaaaatca

SEQ ID NO: 263 - DNA, 19 bp, Artificial
  Probe for Moraxella osloensis
caggtaacgtctaatctaa

SEQ ID NO: 264 - DNA, 19 bp, Artificial
  Probe for Klebsiella pneumonia
atcgatgaggttattaacc

SEQ ID NO: 265 - DNA, 19 bp, Artificial
  Probe for Gemella morbillorum
aaccaacttttaaatatct

SEQ ID NO: 266 - DNA, 19 bp, Artificial
  Probe for Fusobacterium consensus
ctaagaatagttttctgag

SEQ ID NO: 267 - DNA, 19 bp, Artificial
  Probe for Enterococcus faeciu
tttaagagattagcttagc

SEQ ID NO: 268 - DNA, 19 bp, Artificial
  Probe for Enterobacter agglomeran
gtaacattctgatttacga

SEQ ID NO: 269 - DNA, 19 bp, Artificial
  Probe for Corynebacterium consensus
ctaaagtatggtgtcctat

SEQ ID NO: 270 - DNA, 19 bp, Artificial
  Probe for Clostridium clostridiiforme
gaaaacttcatcttaattg

SEQ ID NO: 271 - DNA, 19 bp, Artificial
  Probe for Enterococcus faecalis
agataccgtcaggggacgt

SEQ ID NO: 272 - DNA, 19 bp, Artificial
  Probe for Enterobacter agglomerans
agaactcaagctgccagtt

SEQ ID NO: 273 - DNA, 19 bp, Artificial
  Probe for CNS consensus
tagctcctaataaatggtt

SEQ ID NO: 274 - DNA, 19 bp, Artificial
  Probe for Citrobacter freundi
ccttcctctagtttatcac

SEQ ID NO: 275 - DNA, 19 bp, Artificial
  Probe for Burkholderia cepacia
gtattagaaccaaggattt

SEQ ID NO: 276 - DNA, 19 bp, Artificial
  Probe for Bacteroides fragili
acatactgcactttattct

SEQ ID NO: 277 - DNA, 19 bp, Artificial
  Probe for Staphylococcus aureus
agcagttactctacaattt

SEQ ID NO: 278 - DNA, 19 bp, Artificial
  Probe for Acinetobacter baumannii
taggtattaactaaagtag

SEQ ID NO: 279 - DNA, 19 bp, Artificial
  Probe for Serratia marcescen
caattgatgagcgtattaa

SEQ ID NO: 280 - DNA, 19 bp, Artificial
  Probe for Pseudomonas aeruginosa
actttctccctcaggacgt

SEQ ID NO: 281 - DNA, 19 bp, Artificial
  Probe for Proteus mirabilis
ggtaacgtcaattgataag

SEQ ID NO: 282 - DNA, 19 bp, Artificial
  Probe for PeptoStreptococcus micros
cgtcattatcttctcatag

SEQ ID NO: 283 - DNA, 19 bp, Artificial
  Probe for Moraxella catarrhalis
actaagtatcagaagcaag

SEQ ID NO: 284 - DNA, 19 bp, Artificial
  Probe for Klebsiella pneumonia
tcaatcgatgaggttatta

SEQ ID NO: 285 - DNA, 19 bp, Artificial
  Probe for Gemella morbillorum
tagttactacatatccatt

SEQ ID NO: 286 - DNA, 19 bp, Artificial
  Probe for Fusobacterium consensus
tccgaactaagaatagttt

SEQ ID NO: 287 - DNA, 19 bp, Artificial
  Probe for Enterococcus faecalis
ttatccccctctgatgggt

SEQ ID NO: 288 - DNA, 19 bp, Artificial
  Probe for Enterobacter agglomeran
gatgaagtattaatttcac

(19) United States
(12) Patent Application Publication
(10) Pub. No.: US 2007/0015151 A1
(43) Pub. Date: Jan. 18, 2007

(54) Analytical chip with an array of immobilized specific recognition elements for the determination of clinically relevant bacteria and analytical method based thereon

(75) Inventors: Jacques Schrenzel, Geneve (CH); Patrice Francois, Cran-Grevrier (FR); Yvan Charbonnier, Bernex (CH); Jean Gabriel Jacquet, Vessy (CH); Utinger Dominic, Ettingen (CH); Gerhard Matthias Kresbach, Staufen (DE); Andreas Peter Abel, Basel (CH); Markus Ehrat, Magden (CH)

Correspondence Address: WENDEROTH, LIND & PONACK, L.L.P., 2033 K STREET N. W., SUITE 800, WASHINGTON, DC, 20006-1021, US

(73) Assignee: Hopitaux Univesitaires de Geneve, Geneve (CH)

(21) Appl. No.: 10/530,910

(22) PCT Filed: Sep. 24, 2003
(86) PCT No.: PCT/EP03/10626

§ 371 Date: Apr. 8, 2005

(30) Foreign Application Priority Data
Oct. 9, 2002 (EP) 02022631.2

(51) Int. Cl. C12Q 1/68; G01N 33/554; C12M 1/34; G01N 33/569

(52) U.S. Cl. 435/6; 435/7.32; 435/287.2; 436/518; 977/900

(57) ABSTRACT

The invention is related to an analytical chip for the simultaneous determination of one or more different bacterial 16S-rRNA in a liquid sample comprising—an evanescent field measurement platform, e.g. an optical waveguide, as a solid carrier and—a plurality of specific recognition elements immobilized in discrete measurement areas of known location forming an array of measurement areas on said evanescent field measurement platform, wherein—a multitude (i.e. 2 or more) of different specific recognition elements is immobilized in discrete measurement areas for the recognition and detection of each different 16S-rRNA, different recognition elements being specific for different subsequences of the 16S-rRNA to be detected, which are not directly adjacent and not overlapping in the sequence of said 16S-rRNA, and—said analytical chip is operable for the detection of 16S-rRNA in the evanescent field of the evanescent field measurement platform, without an amplification (e.g. by polymerase chain reaction PCR or linear amplification “T7”) of the polynucleotide sequences contained in the sample. The invention is also related to an analytical method based on the use of said analytical chip.

About 50 different bacteria are responsible for over 90% of current occurrence of bacterial infections causing diseases of humans. Therefore, an analysis platform is highly desirable, that would be operable for analyzing a provided sample simultaneously for one or several bacterial targets in a uniform format, with a short time-to-result all or for a significant part of these bacteria or compounds respectively markers derived therefrom.

Bacterial pathogens are usually identified by time-consuming biochemical testing, e.g. of metabolic products, cell constituents or cell wall constituents, monitoring of bacterial growth in selected culture media, etc. These techniques, requiring the cultivation of the samples, need several hours to days for growth and isolation of the organisms followed by, e.g., measurement of enzymatic activity, of anti-microbial susceptibilities, or of concentrations of metabolites. During this time, a tentative diagnosis is made on the basis of clinical presentation. Patients receive empirical treatments with broad-spectrum antibiotics as immediate medical action. This increases costs and promotes antibiotic resistance, decreasing the usefulness of entire antibiotic families.

Molecular techniques allow rapid and sensitive identification of slow growing (M. tuberculosis) or even non-cultivable bacteria (M. genavense; B. Hirschel, H. R. Chang, N. Mach, P. F. Piguet, J. Cox, J. D. Piguet, M. T. Silva, L. Larsson, P. R. Klatser and J. E. Thole. 1990. “Fatal infection with a novel, unidentified mycobacterium in a man with the acquired immunodeficiency syndrome.” New England. Journal. of. Medicine 323:109-113; E. C. Bottger, A. Teske, P. Kirschner, S. Bost, H. R. Chang, V. Beer and B. Hirschel. 1992. “Disseminated “Mycobacterium genavense” infection in patients with AIDS”. Lancet 340:76-80). However, they require prior pathogen isolation and/or careful sample preparation. With the notable exception of sequencing, these methods can be defined as “closed” and “serial” because each assay identifies only a single specific pathogen.

Nucleic acid probes, labeled with enzymes, antigenic substrates, chemiluminescent moieties, or radioisotopes, can bind with high specificity to complementary sequences of a target nucleic acid. The sensitivity of the assay depends on the size of the probe, the degree of homology with its target and the labeling method. Present technology typically requires cultivation followed by enzymatic amplification by culture or by enzymatic approaches. The latter include thermal cycle strategies such as the polymerase chain reaction (PCR), as well as isothermal strategies such as transcription-mediated amplification (TMA, Accuprobe®, Gen Probe, San Diego, Calif.) or strand displacement amplification (SDA, Becton-Dickinson, New Jersey, N.J.). These target amplification strategies suffer from the possibility of self-contamination, since the product of one reaction serves as template for the next. As organism identification is more reproducible from pure culture or isolated colonies than from direct specimens, results are usually not available the same day.

Instead of amplifying the template itself, alternate strategies amplify signal from the template, reducing the risk of contamination. One such signal amplification strategy uses a proprietary conformation-sensitive endonuclease (Invader™, Third Wave Technologies, Madison, Wis.). This strategy is successful in identifying microorganism species (D. J. Marshall, L. M. Heisler, V. Lyamichev, C. Murvine, D. M. Olive, G. D. Ehrlich, B. P. Neri and M. de Arruda. 1997. <<Determination of hepatitis C virus genotypes in the United States by cleavase fragment length polymorphism analysis.>> Journal. of. Clinical. Microbiology. 35:3156-3162.), antibiotic resistance (S. Sreevatsan, J. B. Bookout, F. M. Ringpis, S. L. Mogazeh, B. N. Kreiswirth, R. R. Pottathil and R. R. Barathur. 1998. <<Comparative Evaluation of Cleavase Fragment Length Polymorphism With PCR-SSCP and PCR-RFLP to Detect Antimicrobial Agent Resistance in Mycobacterium tuberculosis.>> Mol. Diagn. 3:81-91), and mutations (S. Rossetti, S. Englisch, E. Bresin, P. F. Pignatti and A. E. Turco. 1997. <<Detection of mutations in human genes by a new rapid method: cleavage fragment length polymorphism analysis (CFLPA).>> Molecular. & Cellular. Probes. 11:155-160.), all by genotype identification. The strength of the method lies in isothermal detection and absence of amplicon contamination. However, this powerful signal amplification technology does not rival PCR amplification rates and lacks parallelism and automation. Other signal amplification strategies include ligase-chain reaction (LCR, Abbott, Ill.), branched DNA (bDNA, Chiron, Emeryville, Calif.), or label-antibody-label stacks.

Automated analysis of nucleic acid sequences remains a marginal strategy for bacterial identification. Although various detection methods have been automated, all suffer from low sensitivity and require some amplification before detection can take place. To date, most molecular techniques rely on PCR. Automated PCR coupled with EIA detection has been used for the identification of several microorganism (COBAS AMPLICOR™, Roche): M. tuberculosis (S. X. Wang and L. Tay. 1999. <<Evaluation of three nucleic acid amplification methods for direct detection of Mycobacterium tuberculosis complex in respiratory specimens.>> J. Clin. Microbiol. 37:1932-1934.), C. trachomatis (R. Pasternack, P. Vuorinen and A. Miettinen. 1999. <(Comparison of a transcription-mediated amplification assay and polymerase chain reaction for detection of Chlamydia trachomatis in first-void urine.>> Eur. J. Clin. Microbiol. Infect. Dis. 18:142-144.), or viruses like HSV-2 (J. Groen, B. Hersmus, H. G. Niesters, W. Roest, G. van Dijk, W. van der Meijden and A. D. Osterhaus. 1999. <<Evaluation of a fully automated glycoprotein G-2 based assay for the detection of HSV-2 specific IgG antibodies in serum and plasma.>> J. Clin. Virol. 12:193-200.), HIV, hepatitis B or C (W. K. Roth, M. Weber and E. Seifried. 1999. <<Feasibility and efficacy of routine PCR screening of blood donations for hepatitis C virus, hepatitis B virus, and HIV-1 in a blood-bank setting.>> Lancet 353:359-363; A. Doglio, C. Laffont, F. X. Caroli-Bosc, P. Rochet and J. Lefebvre. 1999. <<Second generation of the automated Cobas Amplicor HCV assay improves sensitivity of hepatitis C virus RNA detection and yields results that are more clinically relevant.>> J. Clin. Microbiol. 37:1567-1569.). Once again, these technologies are targeted to and optimized for the detection of only a single suspected microorganism per assay and yield only qualitative results.

Real-time PCR offers interesting improvements to clinical microbiology, including shorter turn-around time and reduced risk of amplicon contamination. On-line fluorescence monitoring of PCR-generated amplicons is achieved, e.g., through molecular beacons (S. Tyagi and F. R. Kramer. 1996. <<Molecular beacons: probes that fluoresce upon hybridization.>> Nature Biotechnology 14:303-308), fluorescence-resonance energy transfer between probes hybridized to adjacent sites on the amplicon (C. A. Gelfand, G. E. Plum, S. Mielewczyk, D. P. Remeta and K. J. Breslauer. 1999. <<A quantitative method for evaluating the stabilities of nucleic acids.>> Proc. Natl. Acad. Sci. U.S.A. 96:6113-6118; N. Ota, K. Hirano, M. Warashina, A. Andrus, B. Mullah, K. Hatanaka and K. Taira. 1997. <<Structural analysis of nucleic acids by using fluorescence resonance energy transfer (FR).>> Nucleic. Acids. Symposium. Series. 207-208), or TaqMan™ probes (C. A. Heid, J. Stevens, K. J. Livak and P. M. Williams. 1996. <<Real time quantitative PCR.>> Genome Research. 6:986-994.). Rugged systems for very rapid field-testing (P. Belgrader, W. Benett, D. Hadley, J. Richards, P. Stratton, R. Jr. Mariella and F. Milanovich. 1999. <<PCR detection of bacteria in seven minutes.>> Science 284:449-450) integrate sample preparation, amplification and detection in the same cartridge (GeneXpert™ System, Cepheid, Sunnyvale, Calif.). This is close to a practical point-of-care instrument. Its major drawback is the need to define the pathogen(s) to be tested and select the appropriate cartridge(s). Thus, an “open diagnosis” would require one specific cartridge for each putative pathogen.

To date, a single commercially available system allows parallel determination of up to 96 different reactions for the same patient using a 96-wells plate format (ABI PRISM 5700/7700, Perkin-Elmer BioSystems, Poster City, Calif.) or a 384-wells plate format (ABI Prism 7900 HT). However, its costs will not permit routine real-time diagnosis testing. Multiplex PCR, using numerous primer pairs and probes within a single reaction tube, can detect nucleic acid fragments from different organisms. Unfortunately, its development is tedious and the modest overall decrease in the total number of wells will not lead to significant cost savings.

16S ribosomal RNA (16S-rRNA) gene sequencing has been used successfully for phylogenetic analysis (M. C. Enright, P. E. Carter, I. A. MacLean and H. McKenzie. 1994. <<Phylogenetic relationships between some members of the genera Neisseria, Acinetobacter, Moraxella, and Kingella based on partial 16S ribosomal DNA sequence analysis.>> International. Journal. of. Systematic. Bacteriology. 44:387-391.) as well as for bacterial identification (MicroSeq® kit, Perkin-Elmer Biosystems, Foster City, Calif.). Using universal primers to amplify discriminant sequences, this method can detect unexpected or even previously unknown pathogens (T. whippeli (D. A. Relman, T. M. Schmidt, R. P. MacDermott and S. Falkow. 1992. <<(Identification of the uncultured bacillus of Whipple's disease.>> New England. Journal. of. Medicine 327:293-301)). However, also because of universal primers, 16S-rRNA sequencing requires a pure culture of the pathogen before DNA extraction or amplification of the genetic material contained in a body fluid sample (such as blood, cerebral spinal fluid, etc.), after sterilization of the sample. PCR and sequencing procedures require an additional 24 h for identification.

Massively parallel hybridization based on a very large number of DNA probes as recognition elements immobilized in discrete measurement areas forming arrays of measurement areas provided on “chips” (usually simple glass or microscope slides), for specific analyte detection, is at present widely advocated as the diagnostic tool of the future.

For example in U.S. Pat. No. 5,445,934 (Affymax Technologies) arrays of oligonucleotides with a density of more than 1000 features on a square centimeter are described and claimed. The excitation and read-out of such arrays is based on classical optical arrangements and methods. The whole array can be illuminated simultaneously, using an expanded excitation light bundle, which, however, results in a relatively low sensitivity, the portion of scattered light being relatively large and scattered light or background fluorescence light from the glass substrate being also generated in those regions, where no oligonucleotides for binding of the analyte are immobilized. In order to limit excitation and detection to the regions of immobilized features and to suppress light generation in the adjacent regions, there is widespread use of confocal measurement arrangements, and the different features are analyzed sequentially by scanning. The consequences, however, are an increased amount of time for the read-out of a large array and a relatively complex optical set-up.

In the international patent application WO 97/29212 a method for identifying organisms, in especial Mycobacterium tuberculosis, based on the hybridization pattern of a sample and its comparison with a reference hybridization pattern, is claimed. Thereby, the nomenclature “hybridization pattern” is understood as the pattern of the locally resolved signals from an array of measurement areas recorded by a detector, as a consequence of the interaction between immobilized oligonucleotides with the sample. Besides others, also optical detection methods using fluorescence labels attached to the target polynucleotides to be detected are described. These optical methods disclosed in WO 97/29212, however, are based on classical optical configurations which cannot provide sufficient sensitivity for analyzing small amount of sample material or for detecting analytes at very low concentrations. As a consequence, in that disclosure it is strongly preferred that a nucleic acid sample is amplified prior to hybridization. Accordingly, no hint is given in that patent application to a simultaneous determination of target polynucleotides from different organisms or bacteria on one chip, but only to a determination of one or more species-specific polymorphisms.

In summary, a number of promising novel strategies are currently being under development to identify pathogens, such as bacteria, based on determination of their nucleic acid sequences. Most of them require bacterial culturing, followed by enzymatic amplification steps, with the prerequisite of knowledge of the target organism respectively sequence. In the context of this invention such a method, wherein the properties of the target analyte have to be exactly known, shall be called a “closed” analysis method, in contrast, in contrast to “open” analysis methods as provided by the present invention not requiring exact knowledge of the complete target sequence (see below). These amplification steps limit the speed of analysis and the possibilities for multiple parallel identification. There is a need for new techniques and methods which do not require such amplification steps, and which allow for the parallel, simultaneous identification of (one or multiple) organisms (bacteria), e.g. by identification of their 16S-rRNA contained in a sample, by a single measurement and its comparison with reference data.

A significant improvement of detection limits can be achieved, when, instead of the classical detection configurations (for example based on epi-fluorescence excitation as used for most scanners), the determination of an analyte is based on its interaction with the evanescent field, which is, for example, associated with light guiding in an optical waveguide, wherein biochemical or biological recognition elements for the specific recognition and binding of the analyte molecules are immobilized on the surface of the waveguide. When a light wave is coupled into an optical waveguide surrounded by optically rarer media, i.e. media of lower refractive index, the light wave is guided by total reflection at the interfaces of the waveguiding layer. In that arrangement, a fraction of the electromagnetic energy penetrates the media of lower refractive index. This portion is termed the evanescent field. The strength of the evanescent field depends to a very great extent on the thickness of the waveguiding layer itself and on the ratio of the refractive indices of the waveguiding layer and of the media surrounding it. In the case of thin waveguides, i.e. layer thicknesses that are the same as or smaller than the wavelength of the light to be guided, discrete modes of the guided light can be distinguished. As an advantage of such methods, the interaction with the analyte is limited to the penetration depth of the evanescent field into the adjacent medium, being of the order of some hundred nanometers, and interfering signals from the depth of the (bulk) medium can be mainly avoided. The first proposed measurement arrangements of this type were based on highly multi-modal, self-supporting single-layer waveguides, such as fibers or plates of transparent plastics or glass, with thicknesses from some hundred micrometers up to several millimeters.

For a further improvement of the sensitivity and simultaneously for an easier manufacturing in mass production, planar thin-film waveguides have been proposed. In the simplest case, a planar thin-film waveguide consists of a three-layer system: support material (substrate), waveguiding layer, superstrate (respectively the sample to be analyzed), wherein the waveguiding layer has the highest refractive index.

Different methods of analyte determination in the evanescent field of lightwaves guided in optical film waveguides can be distinguished. Based on the applied measurement principle, for example, it can be distinguished between fluorescence, or more general luminescence methods, on one side and refractive methods on the other side. In this context methods for generation of surface plasmon renonance in a thin metal layer on a dielectric layer of lower refractive index can be included in the group of refractive methods, if the resonance angle of the launched excitation light for generation of the surface plasmon resonance is taken as the quantity to be measured. Surface plasmon resonance can also be used for the amplification of a luminescence or the improvement of the signal-to-background ratios in a luminescence measurement. The conditions for generation of a surface plasmon resonance and the combination with luminescence measurements, as well as with waveguiding structures, are described in the literature, for example in U.S. Pat. No. 5,478,755, U.S. Pat. No. 5,841,143, U.S. Pat. No. 5,006,716, and U.S. Pat. No. 4,649,280.

In this application, the term “luminescence” means the spontaneous emission of photons in the range from ultraviolet to infrared, after optical or other than optical excitation, such as electrical or chemical or biochemical or thermal excitation. For example, chemiluminescence, bioluminescence, electroluminescence, and especially fluorescence and phosphorescence are included under the term “luminescence”.

In case of the refractive measurement methods, the change of the effective refractive index resulting from molecular adsorption to or desorption from the waveguide is used for analyte detection. This change of the effective refractive index is determined, in case of grating coupler sensors, from changes of the coupling angle for the in- or out-coupling of light into or out of the grating coupler sensor, in case of interferometric sensors from changes of the phase difference between measurement light guided in a sensing branch and a referencing branch of the interferometer.

The aforesaid refractive methods have the advantage, that they can be applied without using additional marker molecules, so-called molecular labels. The disadvantage of these label-free methods, however, is, that the achievable detection limits are limited to pico-up to nanomolar concentration ranges, dependent on the molecular weight of the analyte, due to lower selectivity of the measurement principle, which is not sufficient for many applications of modern trace analysis, for example for diagnostic applications.

For achieving lower detection limits, luminescence-based methods appear as more adequate, because of higher selectivity of signal generation. In this arrangement, luminescence excitation is limited to the penetration depth of the evanescent field into the medium of lower refractive index, i.e. to the immediate proximity of the waveguiding area, with a penetration depth of the order of some hundred nanometers into the medium. This principle is called evanescent luminescence excitation.

By means of highly refractive thin-film waveguides, based on an only some hundred nanometers thin waveguiding film on a transparent support material, the sensitivity could be increased considerably during the last years. In WO 95/33197, for example, a method is described, wherein the excitation light is coupled into the waveguiding film by a relief grating as a diffractive optical element. The isotropically emitted luminescence from substances capable of luminescence, that are located within the penetration depth of the evanescent field, is measured by adequate measurement devices, such as photodiodes, photomultipliers or CCD cameras. The portion of evanescently excited radiation, that has back-coupled into the waveguide, can also be out-coupled by a diffractive optical element, like a grating, and be measured. This method is described, for example, in WO 95/33198.

Configurations based on thin-film waveguides for the (simultaneous) determination of multiple analytes in a supplied sample are described (e.g. in WO 96/35940), the proposed techniques and analysis methods are always related to a unique assignment and application of one biological or biochemical or synthetic recognition element for one particular analyte to be determined. However, no concept is described for the analysis and identification of one analyte (such as 16S-rRNA) by a plurality of recognition elements, upon generation of a signal pattern leading to the identification of the analyte.

The present invention solves the need defined above. It provides an analytical chip and an analytical method based thereon enabling to analyze a provided sample simultaneously for 16S-rRNA from a multitude of different organisms (bacteria). In contrast to all cited prior art, the invention provides the capability of determining not only one, but a multitude (i.e. two or more) organisms, especially bacteria, simultaneously using one analytical chip according to the invention in an inventive analysis method, without the need for a target amplification. The analytical method according to the invention is readily available for automation, using a commercial analytical system (ZeptoREADER™, Zeptosens AG, Witterswil, Switzerland).

The invention is particularly useful for a fast and easy identification of bacteria by genotypic characterization in a provided sample. Due to the advantageous properties of an evanescent field measurement platform as the sensing platform of an analytical chip according to the invention, considerably less steps of sample preparation are required. Identification of a bacterium even in a complex biological sample is enabled. As a consequence of the lower number of required work-up, steps, which are each associated with the risk of the introduction of experimental error, bias and variation, the reliability and confidence into the results, as well as though-put of an analytical method using the analytical chip according to the invention, is considerably increased in comparison to the known methods. As a further consequence, the analytical chip according to the invention allows for a simultaneous quantitative determination of one or more different bacterial 16S-rRNA in a liquid sample, i.e. with an experimental variation of less than 50%, preferably of less than 20%, most preferably of less than 10%. Thereby, the achievable low degree of experimental variation is of course dependent on the amount of available 16S-rRNA to be detected (i.e. a lower variation can be achieved if more of the 16S-rRNA to be detected is available). In contrast to the known analytical equipment and analysis methods based thereon, especially due to the lower number of required work-up steps (especially of biological/and or biochemical work-up steps typically inducing a large variability of measurement results), and the avoidance of enzymatic and signal amplification steps, surprisingly even a quantitative determination of the amount respectively concentration of the one or more different bacteria in the original sample from where the liquid sample containing said one or more different 16S-rRNA have been derived, is enabled.

The invention includes the use of genomic target analytes other than 16S-rRNA for identification of organisms (e.g. bacteria), such as 23S-rDNA, Internal Transmission Sequences (ITS) and the like, as they are known to a person skilled in the art.

The selection of characteristic subsequences of a 16S-rRNA to be detected, and consequently of their complementary recognition elements (=capture probes to be immobilized), may be based, e.g., on sequence information, GC-content, positioning in a stable region (i.e., a region without frequent mutational variations), physico-chemical parameters, such as the melting temperature, and structural information (e.g. about loops, bulges, etc.). The selection can be supported and optimized using statistical and other mathematical methods, such as hierarchical cluster analysis (HCA), principal component analysis (PCA), and artificial neural networks (ANN). These methods are described in the literature.

In addition, the named statistical or mathematical analysis methods, like PCA, HCA, and ANN, are used for optimization and especially reduction or minimization of the set (number) of different capture probes for a certain 16S-rRNA to be detected for identification of the related organism (bacterium).

The interpretation of the results, i.e., the assignment of an observed hybridization or binding pattern, may be based not only on a simple comparison with reference or library data, but supported by the same type of statistical and mathematical methods (e.g. hierarchical cluster analysis, principal component analysis, artificial neural networks, etc.). The results can be utilized for generating data libraries, as well as comparison with data libraries can be performed for identification of an organism (bacterium) based on the measured hybridization or binding patterns. Typically, the mentioned statistical respectively mathematical methods provide a ranking of probabilities of the identity of an organism to be identified with reference organisms, based on the comparison of the actual binding (respectively hybridization) patterns with reference patterns.

A first subject of the invention is an analytical chip for the simultaneous determination of one or more different bacterial 16S-rRNA in a liquid sample

comprising
an evanescent field measurement platform, e.g. an optical waveguide, as a solid carrier and
a plurality of specific recognition elements immobilized in discrete measurement areas of known location forming an array of measurement areas on said evanescent field measurement platform,

wherein
a multitude (i.e. 2 or more) of different specific recognition elements is immobilized in discrete measurement areas for the recognition and detection of each different 16S-rRNA, different recognition elements being specific for different subsequences of the 16S-rRNA to be detected, which are not directly adjacent and not overlapping in the sequence of said 16S-rRNA, and
and said analytical chip is operable for the detection of 16S-rRNA in the evanescent field of the evanescent field measurement platform, without an amplification (e.g. by polymerase chain reaction PCR or linear amplification “T7”) of the polynucleotide sequences contained in the sample.

Under “linear amplification “T7”” it shall be understood a linear amplification using an in vitro transcription initiated by a 17 promoter.

In the spirit of this invention, spatially separated or discrete measurement areas shall be defined by the closed area that is occupied by binding partners, such as polynucleotides, immobilized thereon, for determination of one or multiple analytes in one or multiple samples in a bioaffinity assay, such as a hybridization assay. These areas can have any geometry, for example the form of circles, rectangles, triangles, ellipses etc.

It is preferred that the one or more bacterial 16S-rRNA to be detected are derived from bacteria selected from the group comprising, e.g.: Achromobacter xylosoxidans, Acinetobacter baumannii, Acinetobacter calcoaceticus, Acinetobacter junii, Acinetobacter wolfii, Actinobacillus sp, Actinomyces israelii, Actinomyces meyeri, Actinomyces odontolyticus, Actinomyces sp, Aerococcus viridans, Aeromonas caviae, Aeromonas hydrophilia, Aeromonas sobria, Agrobacterium radiobacter, Alcaligenes denitrificans, Alcaligenes faecalis, Alcaligenes sp, Alcaligenes xylosoxydans, Bacillus sp, Bacteroides bivius, Bacteroides buccae, Bacteroides caccae, Bacteroides denticola, Bacteroides disiens, Bacteroides distasonis, Bacteroides fragilis, Bacteroides oralis, Bacteroides oris, Bacteroides ovatus, Bacteroides stercoris, Bacteroides thetaiotomicron, Bacteroides uniformis, Bacteroides ureolyticus, Bacteroides vulgatus, Bifidobacterium sp, Bordetella bronchiseptica, Brucella melitensis, Burkholderia cepacia, Burkholderia picketti, Burkholderia pseudomallei, Campylobacter coli, Campylobacter fetus, Campylobacter jejuni, Campylobacter sp, Capnocytophaga canimorsus, Capnocytophaga ochracea, Capnocytophaga sp, Chryseomonas luteola, Citrobacter amalonaticus, Citrobacter braakii, Citrobacter diversus, Citrobacter freundii, Citrobacter koseri, Citrobacter sp, Clostridium bifermentans, Clostridium butyricum, Clostridium clostridiiforme, Clostridium paraputrificum, Clostridium perfringens, Clostridium ramosum, Clostridium septicum, Clostridium tertium, Clostridiumi innocuum, Comamonas acidovora, Corynebacterium aquaticum, Corynebacterium bovis, Corynebacterium jeikeium, Corynebacterium minutissimum, Corynebacterium sp, Eikenella corrodens, Empedobacter brevis, Entereococcus casseliflavus, Enterobacter aerogenes, Enterobacter agglomerans, Enterobacter amnigenus, Enterobacter cloacae, Enterococcus avium, Enterococcus durans, Enterococcus faecalis, Enterococcus faecium, Enterococcus gallinarium, Enterococcus raffinosus, Escherichia coli, Eubacterium aerofaciens, Eubacterium lentum, Eubacterium limosum, Flavobacterium breve, Flavobacterium meningosepticum, Flavobacterium sp, Fusobacterium sp, Fusobacterium mortiferum, Fusobacterium necrophorum, Fusobacterium nucleatuim, Fusobacterium varium, Gardnerella vaginalis, Gemella haemolysans, Gemella morbillorum, Gemella sp, Haemophilus aphrophilus, Haemophilus influenzae, Haemophilus parainfluenzae, Haemophilus paraphrophilus, Hafnia alvei, Kingella sp, Klebsiella ornithinolytica, Klebsiella oxytoca, Klebsiella ozaenae, Klebsiella pneunoniae, Kluyvera sp, Lactobacillus acidophilus, Lactobacillus catenaforme, Lactococcus cremoris, Lactococcus lactis, Legionella pneumophila, Leptotrichia buccalis, Leuconostoc sp, Listeria monocytogenes, Moraxella catarrhalis, Moraxella osloensis, Moraxella phenylpyruvica, Moraxella sp, Morganella morganii, Mycobacterium avium, Mycobacterium genavense, Mycobacterium tuberculosis, Mycobacterium avium-intracellulare, Mycoplasma sp, Myroides odoratum, Neisseria cinerea, Neisseria flavescens, Neisseria meningitidis, Neisseria mucosa, Neisseria sp, Neisseria subflava, Nocardia asteroides, Nocardia sp, Ochrobactrum anthropi, Pasteurella multocida, Peptostreptococcus anaerobius, Peptostreptococcus asaccharolyticus, Peptostreptococcus magnus, Peptostreptococcus micros, Peptostreptococcus prevotii, Prevotella bivia, Prevotella buccae, Prevotella loescheii, Propionibacterium acnes, Propionibacterium granulosum, Proteus mirabilis, Proteus penneri, Proteus vulgaris, Providencia rettgeri, Providencia sp, Providencia stuartii, Pseudomonas aeroginosa, Pseudomonas alcaligenes, Pseudomonas diminuta, Pseudomonas fluorescens, Pseudomonas paucimobilis, Pseudomonas putida, Pseudomonas sp, Pseudomonas stutzeri, Pseudomonas vesicularis, Salmonella enteritidis, Salmonella paratyphi, Salmonella typhi, Salmonella typhimurium, Serratia fonticola, Serratia marcescens, Serratia odorifera, Serratia sp, Shigella dysenteria, Shigella flexneri, Shigella sonnei, Sphingomonas paucimobilis, Staphylococcus aureus, Staphylococcus auricularis, Staphylococcus capitis, Staphylococcus caprae, Staphylococcus chromogenes, Staphylococcus cohnii, Staphylococcus epidermidis, Staphylococcus haemolyticus, Staphylococcus hominis, Staphylococcus intermedius, Staphylococcus kloosii, Staphylococcus lugdunensis, Staphylococcus saccharolyticus, Staphylococcus saprophyticus, Staphylococcus sciuri, Staphylococcus simulans, Staphylococcus warneri, Staphylococcus xylosus, Stenotrophomonas maltophilia, Stomatococcus mucilaginosus, Streptococcus acidiminimus, Streptococcus adjacens, Streptococcus agalactiae, Streptococcus anginosus, Streptococcus bovis, Streptococcus canis, Streptococcus constellatus, Streptococcus cremoris, Streptococcus crista, Streptococcus defectivus, Streptococcus dysgalactiae, Streptococcus equinus, Streptococcus equisimilis, Streptococcus intermedius, Streptococcus lactis, Streptococcus mitis, Streptococcus mutans, Streptococcus oralis, Streptococcus pneumoniae, Streptococcus pyogenes, Streptococcus salivarius, Streptococcus sanguis, Streptococcus alpha-hemolyticus, Streptococcus beta-hemolyticus, Veillonella parvula, Veillonella sp, Yersinia enterocolitica, and the like.

Although polynucleotides are the most obvious recognition elements for recognition of and hybridizing with complementary subsequences of a 16S-rRNA to be detected, other possible recognition elements for RNA have been described, which are, for example, based on RNA-ligand interactions, especially on RNA-protein interactions. This is attributed to the observation that unlike DNA, which mostly occurs as a base-paired duplex of complementary strands, RNA is almost always folded from a single strand. Electrostatic repulsion between sections of the highly charged ribose-phosphate backbone are regarded as a driving force for RNA folding. As a consequence, the RNA can assume secondary structures which can be recognized, for example, by proteins (R. A. Zimmermann, et al. 2000. “How ribosomal proteins and rRNA recognize one another”. The ribosome: Structure, function, antibiotics, and cellular interactions. (R. A. Garrett et al. eds.), pp. 93-104, ASM Press, Washington, D.C.) and also by specifically targeted synthetic molecules, like antiviral drugs (D. J. Ecker, R. H. Griffey, 1999. “RNA as a small molecule drug target: doubling the value of genomics.” Drug Discovery Today 4, 420-429) that bind to viral-specific RNA.

Accordingly, for one group of embodiments of an analytical chip according to the invention, it is characteristic that the immobilized specific recognition elements are selected from the group comprising, e.g., natural and synthetically fabricated polynucleotides, polynucleotides with artificial bases and/or artificial carbohydrates, peptides, peptide nucleic acids (“PNA”s), PNA's with artificial bases, Locked nucleic acids® (LNAs) (Exiqon, DK-2950 Vedbaek, Denmark), proteins (e.g. antibodies), ribozymes, and aptamers.

According to the recognition elements, another group of embodiments of the analytical chip can be distinguished, wherein the immobilized specific recognition elements are selected from the group of antibiotics-based, DNA- or RNA-selective recognition elements comprising, e.g., macrolide antibiotics (e.g. erythromycin, azithromycin, streptogramin), aminoglycoside antibiotics (e.g. neomycin, paromomycin, lividomycin, gentamycin), and peptide antibiotics (e.g. thiostreptone, micrococcin) and the like.

Preferred, however, is an embodiment of an analytical chip for the simultaneous determination of one or more different bacterial 16S-rRNA in a liquid sample

comprising
an evanescent field measurement platform, e.g. an optical waveguide, as a solid carrier and
a plurality of polynucleotides immobilized in discrete measurement areas of known location forming an array of measurement areas on said evanescent field measurement platform,

wherein
a multitude (i.e. 2 or more) of different polynucleotides is immobilized in discrete measurement areas for the detection of each different 16S-rRNA, the sequences of the immobilized polynucleotides being essentially complementary to different subsequences of the 16S-rRNA to be detected, which are not directly adjacent and not overlapping in the sequence of said 16S-rRNA, and
and said analytical chip is operable for the detection of 16S-rRNA in the evanescent field of the evanescent field measurement platform, without an amplification (e.g. by polymerase chain reaction PCR or linear amplification “T7”) of the polynucleotide sequences contained in the sample.

“Essential complementarity” between the sequences of the immobilized polynucleotides and subsequences of the 16S-rRNA shall mean, that these sequences are complementary except for no more than 10% mismatches. Accordingly, also bacterial 16S-rRNA with corresponding point mutations in the subsequences to be hybridized can be analyzed with an analytical chip according to the invention and with a corresponding analytical method according to the invention (see below), using said analytical chip. Dependent on the choice of the sequence of the immobilized polynucleotides (capture probes), with respect to the 16S-rRNA to be detected, such mutations may be detected using an analytical chip according to the invention, or they may be disregarded.

In the context of this patent application, “hybridization” between essentially complementary poynucleotides is understood as a special form of binding between a specific recognition element and the analyte (or another specific binding partner), identical to or comparable with the Watson/Crick base pair interaction.

Preferably, the immobilized polynucleotides for the detection of the bacterial 16S-rRNA have a length of 5-500, more preferably of 10-100, most preferably of 10-30 bases.

These immobilized polynucleotides (capture probes) may have similar length (number of base pairs), or different capture probes may also differ in length.

In typical embodiments of the analytical chip according to the invention, the plurality of immobilized polynucleotides comprises 2-20 different polynucleotides which are essentially complementary to different subsequences of the same bacterial 16S-rRNA to be detected.

Due to the high sensitivity provided by analytical chip according to the invention, not requiring an amplification (e.g. by polymerase chain reaction PCR or linear amplification “T7”) of the polynucleotide sequences contained in the sample, the risk of falsifications occurring during the amplification process can be avoided. Therefore, in general a lower number of different immobilized specific recognition elements (respectively of polynucleotides for the preferred embodiments) is required for the unambiguous detection of a certain 16S-rRNA than for known analytical chips, which in general can only be applied in combination with the amplification of the biological material contained in a sample. It is preferred, that the plurality of immobilized polynucleotides (or more generally: of specific recognition elements) comprises less than 10, preferably less than 5 different polynucleotides (respectively specific recognition elements) which are essentially complementary to different subsequences (respectively: can specifically bind to different subsequences) of the same bacterial 16S-rRNA to be detected.

Besides the obvious advantage of reduced costs for the production of an analytical chip with only a small number of specific recognition elements for the target (16S-rRNA) to be determined, further advantages are a sharper level of discrimination whether hybridization (respectively in case of recognition elements other than polynucleotides: whether specific binding) occurred or not, and a less ambiguous result of the comparison of an obtained sample hybridization pattern with reference data. Due to the small required number of capture probes (specific recognition elements in general, polynucleotides in especial), a large number of different 16S-rRNA can be addressed on one and the same analytical chip.

It is well-known that a bacterial 16S-rRNA contains both subsequences that are characteristic for a certain genus and common for all species of that genus, and other subsequences characteristic for a certain or even for a strain. Accordingly, as one possible embodiment of an analytical chip according to the invention the sequences of the multitude of immobilized polynucleotides for detection of a 16S-rRNA are essentially complementary to subsequences indicative for the genus of the bacterium from which said 16S-rRNA to be detected has been derived. Characteristic for other possible embodiments is, that the sequences of the multitude of immobilized polynucleotides for detection of a 16S-rRNA is essentially complementary to subsequences indicative for the species and/or strain of the bacterium from which said 16S-rRNA to be detected has been derived. Still another embodiment is characterized in that the multitude of immobilized polynucleotides for detection of a 16S-rRNA comprises both polynucleotides with a sequence essentially complementary to subsequences indicative for the genus type and polynucleotides with a sequence essentially complementary to the species and/or strain of the bacterium from which said 16S-rRNA to be detected has been derived. The possibility to identify a genus based on selected “signature” sequences allows the identification or exclusion of unknown species that are not part of an established pattern library.

Besides high sensitivity, the evanescent field measurement platform provides to the analytical chip according to the invention as a further advantage that signal generation and detection is confined to the sensing surface and that the risk of interfering signals from the sample matrix in the bulk medium, outside the penetration depth of the evanescent field, is eliminated. As a consequence, a sample to be analyzed can be embedded within almost any medium, and considerably less sample preparation (for example for simplification of the sample matrix) is required compared to analysis on conventional supports like microscope slides, using, e.g., confocal epifluorescence detection. Without restriction of generality, the liquid sample may comprise a complex biological matrix of the group of human and animal cell extracts, extracts of human and animal tissue, such as organ, skin or bone tissue, and of body fluids or their components, such as blood, serum, plasm, lymph, synovia, tear liquid, sweat, milk, sperm, sputum, cerebral spinal fluid, gastric juice, intestinal contents, urine, and stool. For example, the sample may be a clinical sample (e.g. from a patient's blood or body secreta) and may be screened for a variety of bacteria that could be contained therein.

The evanescent field measurement platform may be operable to work with a single total reflection for a launched light ray, like a prism. Then an expanded bundle of essentially parallel light rays would be launched in such a way that it would hit the surface on which an array of measurement areas would be accommodated under an angle matching the condition for total reflection.

“Essentially parallel” shall mean, that the angle of divergence or convergence of a light bundle is not more than 1°.

However, it is much more convenient, if the evanescent field measurement platform can provide multiple points of total reflection, with multiple isolated locations of generation of an evanescent field in the outside medium, or even a continuous evanescent field zone, as it is characteristic for low-mode waveguides.

Therefore, it is strongly preferred if the evanescent field measurement platform comprises an optical waveguide. The optical waveguide may be continuous or be partitioned into discrete waveguiding areas.

It is preferred that the optical waveguide is provided as optical film waveguide with a first optically transparent layer (a) on a second optically transparent layer (b) with lower refractive index than layer (a). “Optical transparency” of a material shall mean that it is transparent at least at the wavelength of an irradiated excitation light, that can have a wavelength between the UV and near-IR spectral region (between 200 nm and 1200 nm). If, as a result of irradiation with excitation light, a luminescence or fluorescence is generated, the material should also be transparent at the wavelength of said luminescence or fluorescence, which also may be between 200 nm and 1200 nm.

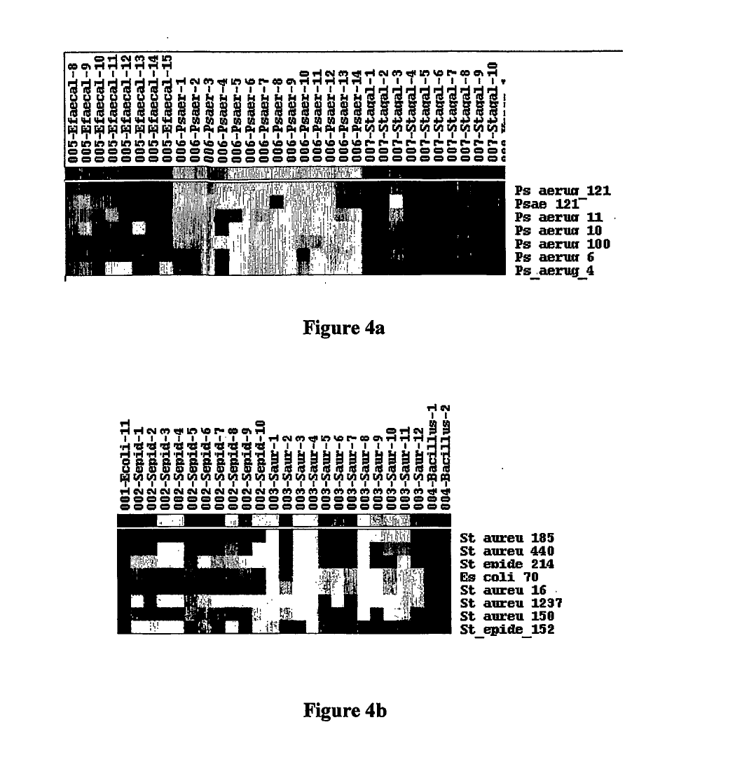

Different embodiments of optical film waveguides, that are suitable for an analytical chip according to the invention as an evanescent field measurement platform, have been described in several international patent applications, such as WO 95/33197, WO 95/33198, WO 96/35940, WO 00/75644, WO 01/43875 (as part of flow cell arrangements), WO 01/79821, and WO 01/88511, that are included in this application in their full entirety. An example of such a thin-film is illustrated in FIG. 4 of WO 01/43875.

It is preferred that the material of the second optically transparent layer (b) comprises, for example, silicates, such as glass or quartz, or a transparent thermoplastic or moldable plastic, preferably of the group comprising polycarbonate, polyimide, or polymethylmethacrylate, or polystyrene.

For generating an evanescent field as strong as possible at the surface of the optically transparent layer (a), it is desirable that the refractive index of the waveguiding, optically transparent layer (a) is significantly higher than the refractive index of the adjacent layers. It is especially advantageous, if the refractive index of the first optically transparent layer (a) is higher than 1.8.

The first optically transparent layer (a) can comprise, for example, TiO₂, ZnO, Nb₂O₅, Ta₂O₅, HfO₂, or ZrO₂. It is especially preferred, if the first optically transparent layer (a) comprises TiO₂, Ta₂O₅ or Nb₂O₅.

Besides the refractive index of the waveguiding optically transparent layer (a), its thickness is the second important parameter for the generation of an evanescent field as strong as possible at the interfaces to adjacent layers with lower refractive index. With decreasing thickness of the waveguiding layer (a), the strength of the evanescent field increases, as long as the layer thickness is sufficient for guiding at least one mode of the excitation wavelength. Thereby, the minimum “cut-off” layer thickness for guiding a mode is dependent on the wavelength of this mode. The “cut-off” layer thickness is larger for light of longer wavelength than for light of shorter wavelength. Approaching the “cut-off” layer thickness, however, also unwanted propagation losses increase strongly, thus setting additionally a lower limit for the choice of the preferred layer thickness.

Preferred are layer thicknesses of the optically transparent layer (a) allowing for guiding only one to three modes at a given excitation wavelength. Especially preferred are layer thicknesses resulting in monomodal waveguides for this given excitation wavelength. It is understood that the character of discrete modes of the guided light does only refer to the transversal modes.

Resulting from these requirements, the thickness of the first optically transparent layer (a) is preferably between 40 and 300 nm. It is especially advantageous, if the thickness of the first optically transparent layer (a) is between 100 and 200 nm.

The amount of the propagation losses of a mode guided in an optically waveguiding layer (a) is determined to a large extent by the surface roughness of a supporting layer below and by the absorption of chromophores which might be contained in this supporting layer, which is, additionally, associated with the risk of excitation of unwanted luminescence in this supporting layer, upon penetration of the evanescent field of the mode guided in layer (a) into this supporting layer. Furthermore, thermal stress can occur due to different thermal expansion coefficients of the optically transparent layers (a) and (b). In case of a chemically sensitive optically transparent layer (b), consisting for example of a transparent thermoplastic plastics, it is desirable to prevent a penetration, for example through micro pores in the optically transparent layer (a), of solvents that might attack layer (b).

Therefore, it is advantageous, if an additional optically transparent layer (b′) with lower refractive index than and in contact with layer (a), and with a thickness of 5 nm-10 000 nm, preferably of 10 nm-1000 nm, is located between the optically transparent layers (a) and (b). The purpose of the intermediate layer is a reduction of the surface roughness below layer (a) or a reduction of the penetration of the evanescent field, of light guided in layer (a), into the one or more layers located below or an improvement of the adhesion of layer (a) to the one or more layers located below or a reduction of thermally induced stress within the optical sensor platform or a chemical isolation of the optically transparent layer (a) from layers located below, by sealing of micro-pores in layer (a) against the layers located below.

The in-coupling of excitation light into the optically transparent layer (a), to the measurement areas, can be performed using one or more optical in-coupling elements from the group comprising prism couplers, evanescent couplers comprising joined optical waveguides with overlapping evanescent fields, front face (distal end) couplers with focusing lenses arranged in front of a front face (distal end) of the waveguiding layer, and grating couplers. Thereby it is preferred that the in-coupling is performed using one or more grating structures (c) (acting as grating couplers), that are formed in the optically transparent layer (a).

Furthermore it is preferred, that out-coupling of light guided in the optically transparent layer (a) is performed using grating structures (c′) that are formed in the optically transparent layer (a). Out-coupling of light guided in layer (a), at defined locations on the analytical chip, can, for example be beneficial to avoid reflections at the distal or lateral ends of the chip, which could lead to interference with the signals generated in the region of the measurement areas.

Grating structures (c) and (c′) formed in the optically transparent layer (a) can have the same or different periodicity and may be arranged in parallel or not in parallel to one another.

Grating structures (c) and (c′) can interchangeably be used as in-coupling and/or out-coupling gratings.

For given refractive indices of the waveguiding, optically transparent layer (a) and of the adjacent layers, the resonance angle for in-coupling of the excitation light, according to a well-defined resonance condition, is dependent on the diffraction order to be in-coupled, on the excitation wavelength and on the grating period. In-coupling of the first diffraction order is advantageous for increasing the in-coupling efficiency. Besides the number of the diffraction order, the grating depth is important for the amount of the in-coupling efficiency. As a matter of principle, the coupling efficiency increases with increasing grating depth. The process of out-coupling being completely reciprocal to the in-coupling, however, the out-coupling efficiency increases simultaneously, resulting in an optimum for the excitation of luminescence in a measurement area located on or adjacent to the grating structure (c), the optimum being dependent on the geometry of the measurement areas and of the launched excitation light bundle. Based on these boundary conditions, it is advantageous, if the grating (c) has a period of 200 nm-1000 nm and a modulation depth of 3 nm-100 nm, preferably of 10 nm-30 nm.

Furthermore, it is preferred that the ratio of the modulation depth of the grating to the thickness of the first optically transparent layer (a) is equal or smaller than 0.2.

It is also preferred that the grating structure (c) is a relief grating with a rectangular, triangular or semi-circular profile or a phase or volume grating with a periodic modulation in the essentially planar, optically transparent layer (a).

For most applications it is preferred, that the grating structure (c) is a diffractive grating with a uniform period. For some special applications, however, for example for in-coupling of light of different excitation wavelengths from different light sources, it can be advantageous if the grating structure (c) is a multi-diffractive grating.

For the geometrical arrangement of the arrays of measurement areas on an evanescent field measurement platform provided with in-coupling gratings, there are several possibilities. As one possible embodiment, one or more measurement areas of an array of measurement areas are provided on a grating structure (c) or (c′).

As another possible embodiment, several or all arrays of measurement areas are provided on a common grating structure (c) or (c′). Evanescent field measurement platforms of this type, with one or more grating structures covering extended parts of the surface, with arrays of measurement areas provided thereon, have been described in more detail, with further embodiments that are also suitable for an analytical chip according to the present invention, in the international patent application WO 00/75644. These embodiments are especially advantageous when a very high surface density of measurement areas is desired, as the propagation (and hence possible optical cross-talk within the waveguiding layer (a)) of guided light in a direction perpendicular to the grating lines is limited to rather short distances, controllable mainly by the grating depth. As a special embodiment of this type, a superposition of two or more gratings with equal or different grating periods may be provided, wherein the grating lines preferably are oriented other than in parallel, for example perpendicular to each other in case of two superimposed gratings.

Characteristic for a third type of embodiments of an analytical chip according to the invention is, that arrays of measurement areas are provided adjacent to or between grating structures (c) or (c′).

There are many methods for the deposition of biological or biochemical or synthetic recognition elements, such as polynucleotides, on the optically transparent layer (a). For example, the deposition can be performed by physical adsorption or electrostatic interaction. In general, the orientation of the recognition elements is then of statistic nature. Additionally, there is the risk of washing away a part of the immobilized recognition elements, if the sample containing the analyte and reagents applied in the analysis process have a different composition. Therefore, it can be advantageous, if an adhesion-promoting layer (f) is deposited on the optically transparent layer (a), for immobilization of biological or biochemical or synthetic recognition elements. This adhesion-promoting layer should be transparent as well. In especial, the thickness of the adhesion-promoting layer should not exceed the penetration depth of the evanescent field out of the waveguiding layer (a) into the medium located above. Therefore, the adhesion-promoting layer (a) should have a thickness of less than 200 nm, preferably of less than 20 nm.

The adhesion-promoting layer can comprise, for example, chemical compounds as known in the art, e.g. of the group comprising silanes, epoxides, functionalized, charged or polar polymers and “self-organized passive or functionalized mono- or multilayers”, alkyl phosphates or alkyl phosphonates, and multifunctional block copolymers, such as poly(L)lysine/polyethylene glycols, and the like.

Laterally separated measurement areas can be generated by laterally selective deposition of biological or biochemical or synthetic recognition elements, such as polynucleotides, on the evanescent field measurement platform. Thereby, one or more deposition methods of the group of methods comprising “ink jet spotting”, mechanical spotting by means of pin, pen or capillary, “micro contact printing”, fluidically contacting the measurement areas with the biological or biochemical or synthetic recognition elements upon their supply in parallel or crossed micro channels, upon exposure to pressure differences or to electric or electromagnetic potentials, can be applied.

In many cases the sensitivity of an analytical method is limited by signals caused by so-called nonspecific binding, i.e. by signals caused by the binding of the analyte or of other components applied for analyte determination or of compounds of the sample matrix, which are not only bound in the area of the provided immobilized biological or biochemical or synthetic recognition elements (e.g. polynucleotides), but also in areas of an evanescent field measurement platform that are not occupied by these recognition elements, for example upon hydrophobic adsorption or electrostatic interactions. Therefore, it is advantageous, if compounds, that are “chemically neutral” towards the analyte and/or towards the sample matrix, are deposited between the laterally separated measurement areas, in order to minimize nonspecific binding or adsorption. As “chemically neutral” compounds such components are called, which themselves do not have binding sites for the recognition and binding of the analyte or of an analogue of the analyte or of a further binding partner in a multi-step assay (and also for compounds of the sample matrix) and which prevent, due to their presence, the access of the analyte or of its analogue or of the further binding partners (or of compounds of the sample matrix) to the surface of the evanescent field measurement platform. Such a “chemically neutral” compound should also minimize nonspecific adhesion to the surface areas where it is deposited.

It is of large advantage, if the adhesion-promoting layer is “chemically neutral” towards compounds other than the recognition elements for the analytes contained in the sample, i.e., reduces nonspecific interaction with these compounds. For example, multifunctional block copolymers, such as poly(L)lysine/polyethylene glycols of adequate grafting ratio, are characterized by this favorable property.

For more general cases, where a minimization of nonspecific binding effects cannot be provided by an adhesion-promoting layer, it is preferred, that compounds which are “chemically neutral” towards the analytes and/or towards other compounds contained in the sample matrix, preferably of the groups comprising, for example, albumines, especially bovine serum albumine or human serum albumine, fragmentated natural or synthetic DNA, such as from herring or salmon sperm, not hybridizing with polynuleotides to be analyzed, or uncharged but hydrophilic polymers, such as polyethyleneglycols or dextranes, are deposited between the laterally separated measurement areas.

On an analytical chip according to the invention, a very large total number of discrete measurement areas can be provided. It is possible to arrange more than 10,000, even up to 1,000,000 measurement areas in 2-dimensional arrangement. A single, individual measurement area can typically have an area between 0.001 mm²-6 mm², whereby different measurement areas can have different size.

The measurement areas can be provided at a density of more than 10, preferably of more than 100, most preferably of more than 1000 measurement areas per square centimeter.

Characteristic for another preferred embodiment of an analytical chip according to the invention is, that the surface with the discrete measurement areas with immobilized polynucleotides forms the inner bottom surface of one or more sample compartments for receiving one or more samples to be analyzed for 16S-rRNA.

Thereby, it is preferred, that the one or more sample compartments are designed to accommodate a sample volume of less than 50 μl each, and that the inner bottom surface of a sample compartment is larger than 10 mm². Different embodiments of sample compartments, that are adequate to be formed with an analytical chip according to the invention, are described in the international patent applications WO 01/13096 and WO 01/43875, which are therefore incorporated in this patent application in their full entirety. Embodiments with a reservoir connected to the outlet of a flow cell, to receive exiting liquid, as described in WO 01/43854, appear especially useful, when sequentially several reagents or washing solutions have to be flown over the surface carrying the immobilized specific recognition elements (e.g. polynucleotides) and eventually 16S-rRNA bound respectively hybridized with them.

Grating structures (c) and optional addition grating structures (c′) may be located within a sample compartment.

If the parts of the side walls touching the surface of the analytical chip, or a connecting material, such as a glue, between these side walls and the analytical chip, are optically transparent (in the sense as defined above), then the grating structures (c) and optional additional grating structures (c′) may also be located outside the sample compartments.

It is advantageous if the grating lines are oriented essentially in parallel to a pair of the side walls of the sample compartments, in order to reduce disturbing effects of reflections or scattering, especially when the grating structures are located outside the sample compartments. The grating structures (c) or (c′) can be limited in their lateral extension on the analytical chip surface to the length of the parallel side walls of the sample compartments. However, they can also extend over the range of multiple or all sample compartments, for example along the whole width of an analytical chip (as defined) in the Example I.A.1 of this patent application.

For consecutive measurement of different arrays of measurement areas, it is of large advantage if the conditions for coupling of excitation light into an evanescent field measurement platform provided with an in-coupling grating vary only very little over extended distances, for example, if the coupling angle varies less than by 0.1°/cm in parallel to the grating lines. Evanescent field measurement platforms of such a high precision have been described in the international patent applications WO 01/55691 and WO 01/55760, which are incorporated in this patent application.

Another subject of the invention is an analytical method for the simultaneous determination of one or more different bacterial 16S-rRNA in a liquid sample, comprising providing an analytical chip comprising
an evanescent field measurement platform, e.g. an optical waveguide, as a solid carrier and
a plurality of specific recognition elements immobilized in discrete measurement areas of known location forming an array of measurement areas on said evanescent field measurement platform,

wherein
a multitude (i.e. 2 or more) of different specific recognition elements is immobilized in discrete measurement areas for the recognition and detection of each different 16S-rRNA, different recognition elements being specific for different subsequences of the 16S-rRNA to be detected, which are not directly adjacent and not overlapping in the sequence of said 16S-rRNA,
a liquid sample, not being subjected to an amplification (e.g. by polymerase chain reaction PCR or linear amplification “T7”) of the polynucleotide sequences contained therein, is brought into contact with the array under conditions allowing for binding (respectively hybridization) of 16S-rRNA contained in the sample with the corresponding specific recognition elements immobilized in the measurement areas
changes of electro-optical signal caused by a successful binding (respectively hybridization) on the measurement areas of the evanescent field measurement platform are measured with one or more detectors, and
the presence of a bacterium to be detected is determined from the whole of signals from those measurement areas occupied by immobilized specific recognition elements dedicated for the specific detection of said bacterium.

It is characteristic for the analytical method according to the invention, that it requires a lower number of required work-up steps, which are each associated with the risk of the introduction of experimental error and variation, than the known methods. As a consequence, the reliability and confidence into the results of an analytical method according to the invention, is considerably increased in comparison to the known methods. As a further consequence, the analytical chip according to the invention allows for a simultaneous quantitative determination of one or more different bacterial 16S-rRNA in a liquid sample, i.e. with an experimental variation of less than 50%, preferably of less than 20%, most preferably of less than 10%. Thereby, the achievable low degree of experimental variation is of course dependent on the amount of available 16S-rRNA to be detected (i.e. a lower variation can be achieved if more of the 16S-rRNA to be detected is available). In contrast to the known analytical equipment and analysis methods based thereon, especially due to the lower number of required work-up steps (especially of biological/and or biochemical work-up steps typically inducing a large variability of measurement results) even a quantitative determination of the amount respectively concentration of the one or more different bacteria in the original sample from where the liquid sample containing said one or more different 16S-rRNA have been derived, is enabled.

It is preferred that the one or more bacterial 16S-rRNA to be detected are derived from bacteria selected from the group comprising e.g.: Achromobacter xylosoxidais, Acinetobacter baumannii, Acinetobacter calcoaceticus, Acinetobacter junii, Acinetobacter wolfii, Actinobacillus sp, Actinomyces israelii, Actinomyces meyeri, Actinomyces odontolyticus, Actinomyces sp, Aerococcus viridans, Aeromonas caviae, Aeromonas hydrophilia, Aeromonas sobria, Agrobacterium radiobacter, Alcaligenes denitrificans, Alcaligenes faecalis, Alcaligenes sp, Alcaligenes xylosoxydans, Bacillus sp, Bacteroides bivius, Bacteroides buccae, Bacteroides caccae, Bacteroides denticola, Bacteroides disiens, Bacteroides distasonis, Bacteroides fragilis, Bacteroides oralis, Bacteroides oris, Bacteroides ovatus, Bacteroides stercoris, Bacteroides thetaiotomicron, Bacteroides uniformis, Bacteroides ureolyticus, Bacteroides vulgatus, Bifidobacterium sp, Bordetella bronchiseptica, Brucella melitensis, Burkholderia cepacia, Burkholderia picketti, Burkholderia pseudomallei, Campylobacter coli, Campylobacter fetus, Campylobacter jejuni, Campylobacter sp, Capnocytophaga canimorsus, Capnocytophaga ochracea, Capnocytophaga sp, Chryseomonas luteola, Citrobacter amalonaticus, Citrobacter braakii, Citrobacter diversus, Citrobacter freundii, Citrobacter koseri, Citrobacter sp, Clostridium bifermentans, Clostridium butyricum, Clostridium clostridiiforme, Clostridium paraputrificum, Clostridium perfringens, Clostridium ramosum, Clostridium septicum, Clostridium tertium, Clostridiumi innocuum, Comamonas acidovora, Corynebacterium aquaticum, Corynebacterium bovis, Corynebacterium jeikeium, Corynebacterium minutissimum, Corynebacterium sp, Eikenella corrodens, Empedobacter brevis, Enterococcus casseliflavus, Enterobacter aerogenes, Enterobacter agglomerans, Enterobacter amnigenus, Enterobacter cloacae, Enterococcus avium, Enterococcus durans, Enterococcus faecalis, Enterococcus faecium, Enterococcus gallinarium, Enterococcus raffinosus, Escherichia coli, Eubacterium aerofaciens, Eubacterium lentum, Eubacterium limosum, Flavobacterium breve, Flavobacterium meningosepticum, Flavobacterium sp, Fusobacterium sp, Fusobacterium mortiferum, Fusobacterium necrophorum, Fusobacterium nucleatum, Fusobacterium varium, Gardnerella vaginalis, Gemella haemolysans, Gemella morbillorum, Gemella sp, Haemophilus aphrophilus, Haemophilus influenzae, Haemophilus parainfluenzae, Haemophilus paraphrophilus, Hafnia alvei, Kingella sp, Klebsiella ornithinolytica, Klebsiella oxytoca, Klebsiella ozaenae, Klebsiella pneumoniae, Kluyvera sp, Lactobacillus acidophilus, Lactobacillus catenaforme, Lactococcus cremoris, Lactococcus lactis, Legionella pneunmophila, Leptotrichia buccalis, Leuconostoc sp, Listeria monocytogenes, Moraxella catarrhalis, Moraxella osloensis, Moraxella phenylpyruvica, Moraxella sp, Morganella morganii, Mycobacterium avium, Mycobacterium genavense, Mycobacterium tuberculosis, Mycobacterium avium-intracellulare, Mycoplasma sp, Myroides odoratum, Neisseria cinerea, Neisseria flavescens, Neisseria meningitidis, Neisseria mucosa, Neisseria sp, Neisseria subflava, Nocardia asteroides, Nocardia sp, Ochrobactrum anthropi, Pasteurella multocida, Peptostreptococcus anaerobius, Peptostreptococcus asaccharolyticus, Peptostreptococcus magnus, Peptostreptococcus micros, Peptostreptococcus prevotii, Prevotella bivia, Prevotella buccae, Prevotella loescheii, Propionibacterium acnes, Propionibacterium granulosum, Proteus mirabilis, Proteus penneri, Proteus vulgaris, Providencia rettgeri, Providencia sp, Providencia stuartii, Pseudomonas aeroginosa, Pseudomonas alcaligenes, Pseudomonas diminuta, Pseudomonas fluorescens, Pseudomonas paucimobilis, Pseudomonas putida, Pseudomonas sp, Pseudomonas stutzeri, Pseudomonas vesicularis, Salmonella enteritidis, Salmonella paratyphi, Salmonella typhi, Salmonella typhimurium, Serratia fonticola, Serratia marcescens, Serratia odorifera, Serratia sp, Shigella dysenteria, Shigella flexneri, Shigella sonnei, Sphingomonas paucimobilis, Staphylococcus aureus, Staphylococcus auricularis, Staphylococcus capitis, Staphylococcus caprae, Staphylococcus chromogenes, Staphylococcus cohnii, Staphylococcus epidermidis, Staphylococcus haemolyticus, Staphylococcus hominis, Staphylococcus intermedius, Staphylococcus kloosii, Staphylococcus lugdunensis, Staphylococcus saccharolyticus, Staphylococcus saprophyticus, Staphylococcus sciuri, Staphylococcus simulais, Staphylococcus warneri, Staphylococcus xylosus, Stenotrophomonas maltophilia, Stomatococcus mucilaginosus, Streptococcus acidiminimus, Streptococcus adjacens, Streptococcus agalactiae, Streptococcus anginosus, Streptococcus bovis, Streptococcus canis, Streptococcus constellatus, Streptococcus cremoris, Streptococcus crista, Streptococcus defectivus, Streptococcus dysgalactiae, Streptococcus equinus, Streptococcus equisimilis, Streptococcus intermedius, Streptococcus lactis, Streptococcus mitis, Streptococcus mutans, Streptococcus oralis, Streptococcus pneumoniae, Streptococcus pyogenes, Streptococcus salivarius, Streptococcus sanguis, Streptococcus alpha-hemolyticus, Streptococcus beta-hemolyticus, Veillonella parvula, Veillonella sp, Yersinia enterocolitica, and the like.

For one group of embodiments of an analytical method according to the invention, it is characteristic that the immobilized specific recognition elements are selected from the group comprising, e.g., natural and synthetically fabricated polynucleotides, polynucleotides with artificial bases and/or artifical carbohydrates, peptides, peptide nucleic acids (“PNA”s), PNA's with artificial bases, locked nucleic acids® (LNAs, DK-2950 Vedbaek, Denmark), proteins (e.g. antibodies), ribozymes, and aptamers.

According to the recognition elements, another group of embodiments of the analytical method can be distinguished, wherein the immobilized specific recognition elements are selected, from the group of antibiotics-based recognition elements comprising, e.g., macrolide antibiotics (e.g. erythromycin, azithromycin, streptogramin), aminoglycoside antibiotics (e.g. neomycin, paromomycin, lividomycin, gentamycin), and peptide antibiotics (e.g. thiostreptone, micrococcin).

Preferred, however, is an embodiment of the analytical method for the simultaneous determination of one or more different bacterial 16S-rRNA in a liquid sample, comprising providing an analytical chip comprising
an evanescent field measurement platform, e.g. an optical waveguide, as a solid carrier and
a plurality of polynucleotides immobilized in discrete measurement areas of known location forming an array of measurement areas on said evanescent field measurement platform,

wherein
a multitude (i.e. 2 or more) of different polynucleotides is immobilized in discrete measurement areas for the detection of each different 16S-rRNA, the sequences of the immobilized polynucleotides being essentially complementary to different subsequences of the 16S-rRNA to be detected, which are not directly adjacent and not overlapping in the sequence of said 16S-rRNA,
a liquid sample, not being subjected to an amplification (e.g. by polymerase chain reaction PCR or linear amplification “T7”) of the polynucleotide sequences contained therein, is brought into contact with the array under conditions allowing a hybridization of 16S-rRNA contained in the sample with essentially complementary polynucleotides immobilized in the measurement areas
changes of electro-optical signal caused by a successful hybridization on the measurement areas of the evanescent field measurement platform are measured with one or more detectors, and
the presence of a bacterium to be detected is determined from the whole of signals from those measurement areas occupied by immobilized polynucleotides dedicated for the specific detection of said bacterium.

Potential target sequences of 16S-rRNA to be derived from the above listed bacteria have been published to a large extent already in the literature. Otherwise they can be determined by known sequencing methods. For obtaining an unambiguous hybridization pattern (respectively “binding pattern”=pattern of the binding signals when using specific recognition elements other than polynucleotides) for a certain 16S-rRNA to be detected, an optimum choice of the target subsequences to be detected is of high importance. For example, if a certain genus shall be detected, then subsequences will be selected which are characteristic for that genus of bacteria and common to all its species or strains, whereas for detection of a certain species subsequences characteristic for only that species should be chosen.

Preferably, immobilized polynucleotides for the detection of the bacterial 16S-rRNA have a length of 5-500, preferably of 10-100 bases.

In typical embodiments of the analytical method according to the invention, the plurality of immobilized polynucleotides comprises 2-20 different polynucleotides which are essentially complementary to different subsequences of the same bacterial 16S-rRNA to be detected.

Due to the high sensitivity provided by an analytical chip according to the invention, not requiring an amplification, in general a lower number of different immobilized polynucleotides (or more generally: of specific recognition elements) is required for the unambiguous detection of a certain 16S-rRNA than for known analytical chips, which in general can only be applied in combination with the amplification of the biological material contained in a sample. It is preferred, that the plurality of immobilized polynucleotides (more generally: of immobilized specific recognition elements) comprises less than 10, preferably less than 5 different polynucleotides (more generally: specific recognition elements) which are essentially complementary to (respectively can bind to) different subsequences of the same bacterial 16S-rRNA to be detected.

Thereby, it is preferred that bacterial genus and/or species and/or strain are determined with a plurality of less than 10, preferably of less than 5 different immobilized polynucleotides (more generally: specific recognition elements), that hybridize specifically with (respectively bind specifically to) subsequences of the 16S-rRNA of said genus or species or strain.

It can be advantageous, for example, to reduce diffusion times of the target analyte towards the immobilized specific recognition elements (e.g. polynucleotides), if the bacterial 16S-rRNA to be detected is fragmented into strands of less than 500, preferably of less than 200 base pairs length.

It is preferred that the evanescent field measurement platform, as the basis for the analytical chip used in the analytical method according to the invention comprises an optical waveguide. Thereby, the optical waveguide can be continuous or partitioned into discrete waveguiding areas.

Especially preferred is, if the optical waveguide is an optical film waveguide with a first optically transparent layer (a) on a second optically transparent layer (b) with lower refractive index than layer (a).

For the further embodiments of an evanescent field measurement platform to be applied in an analytical method according to the invention, the same preferences of different embodiments are valid as described above for the evanescent field measurement platform as part of an analytical chip according to the invention.

The high sensitivity provided to the analytical method according to the invention by the favorite properties of the evanescent field can further be improved, if the resulting signals from the arrays of measurement areas, representing specific binding patterns, are analyzed by special mathematical and/or statistical methods dedicated for improvement of signal-to-noise ratios. Preferred such mathematical and/or statistical methods take advantage of additional input that is known about the system, but cannot directly deduced from the raw data (e.g., the expectation of the occurrence of mutations or of increased or decreased occurrence of an analyte leading to resulting differences between signal patterns to be observed). Examples of such methods, for the detection of mutations, are described in U.S. Pat. Nos. 6,136,541 and 6,142,681, which are incorporated hereby in this application in their full entirety. The use of adequate methods described therein for an improvement of signal-to-noise ratios is part of the analytical method according to the invention.

For the detection of the binding of bacterial 16S-rRNA to the corresponding measurement areas, where their specific recognition elements (e.g. polynucleotides) are immobilized, the change of the so-called effective refractive index resulting from molecular adsorption to or desorption from the waveguide can be used for analyte detection. This change of the effective refractive index is determined, in case of grating coupler sensors (when the arrays of measurement areas are located on a coupling grating), from changes of the coupling angle for the in- and/or out-coupling of light into or out of the grating coupler sensor. In case of interferometric sensors, analyte binding can be determined from changes of the phase difference between measurement light guided in a sensing branch and a referencing branch of the interferometer.

The aforesaid refractive methods have the advantage, that they can be applied without using additional marker molecules, so-called molecular labels. The disadvantage of these label-free methods, however, is, that the achievable detection limits are limited to pico-up to nanomolar concentration ranges, dependent on the molecular weight of the analyte, due to lower selectivity of the measurement principle, which is not sufficient for many applications of modern trace analysis, which can be disadvantageous especially for diagnostic applications.

For achieving still lower detection limits, luminescence-based methods appear as more adequate, because of higher selectivity of signal generation. Therefore, it is preferred that the detection of the presence of bacterial 16S-rRNA is based on the change of one or more luminescences, preferably of one or more fluorescences. Thereby, the luminescence (fluorescence) used for analyte detection is generated by luminescence (fluorescence) labels, which are bound to or associated with the 16S-rRNA to be detected.

It is preferred that said labels are bound to polynucleotides (in especial 16S-rRNA) to be determined in a sample by a chemical (non-enzymatic) conjugation method. The labels may, for example, be added directly to the original sample to be analyzed and can thus be bound directly to the single-stranded nucleic acid (16S-rRNA), without the necessity of a transcription process. For example, the labeling can be performed upon end-labeling of the nucleic acid. Preferably, these labels have excitation and emission wavelengths between 250 nm and 1100 nm. The labels can be selected from luminescent, functionalized or intercalating dyes (a large variety of them being well-known in the literature), and luminescent, functionalized nanoparticles (“quantum dots”, see: W. C. W. Chan and S. Nie, “Quantum dot bioconjugates for ultrasensitive nonisotopic detection”, Science 281 (1998) 2016-2018).

It is preferred, that in-coupling of excitation light into the optically transparent, waveguiding layer (a) of an optical film-waveguide as evanescent field measurement platform used for the analytical method according to the invention, towards the measurement areas located thereon, is performed using one or more grating structures (c), that are formed in the optically transparent layer (a).

Characteristic for the analytical method according to the invention is, that a pattern of said changes of electro-optical signal caused by a successful hybridization of a multitude of immobilized polynucleotides, in different measurement areas, dedicated for the detection of one or more 16S-rRNA, (“sample hybrization pattern” of said 16S-rRNA) to be determined in a sample, is established and recorded. When using specific recognition elements other than polynucleotides (such as proteins), a “sample binding pattern” of said 16S-rRNA is established and recorded.

Preferably, also a “reference hybridization pattern” (respectively a “reference binding pattern” when using specific recognition elements other than polynucleotides) is established and recorded by bringing a liquid sample containing a known amount of one or more different known 16S-rRNA into contact with said analytical chip under conditions allowing for hybridization (respectively binding) between said known 16S-rRNA and the corresponding multitudes of complementary immobilized polynucleotides (more generally: specific recognition elements). Said “reference hybridization patterns” (respectively “reference binding pattern”) are typically stored in a data library.

The sample and the reference hybridization pattern (respectively sample and reference binding pattern) can be established, for example by bringing the sample and the reference probe into contact with the same array of measurement areas (e.g. simultaneously or sequentially using labels with different emission wavelengths and optionally also different excitation wavelengths) and measuring and recording the resulting signal intensities. Especially when a high reproducibility of the array fabrication of the analytical chip with the one or more arrays of measurement areas provided thereon is established, the hybridization (respectively binding) patterns of a sample and a reference can also be determined on different arrays, which are then preferably part of the same analytical chip. For this embodiment, preferably the same label is used both for the sample and for the reference.

Preferably, the obtained “sample hybridization patterns” and “reference hybridization patterns” (respectively “sample binding pattern” and “reference binding pattern”) are stored in a data format that is compatible with the format of existing data libraries. Thus, also published data can be used as “reference hybridization patterns” (respectively “reference binding patterns”).

As a typical embodiment of an analytical method according to the invention, 16S-rRNA contained in a sample are determined by comparison of a sample hybridization (respectively binding) pattern and one or more reference hybridization (respectively binding) patterns, upon determining the degree of agreement between said sample hybridization (respectively binding) pattern and said reference hybridization (respectively binding) patterns.

The comparison can be based, for example, on normalized signal intensities or on the difference or ratio of a sample hybridization (respectively binding) pattern and a reference hybridization (respectively binding) pattern. It is important to note that the assignment of the experimentally observed hybridization (respectively binding) patterns to 16S-rRNA of a certain genus or species or strain is based on the degree of agreement with a reference pattern and not on absolute signal patterns. As a consequence, knowledge of all the subsequences of the 16S-rRNA is not required.

Said degree of agreement between said sample hybridization (respectively binding) pattern and said reference hybridization (respectively binding) patterns can be determined by statistical methods or by other mathematical methods, like hierarchical cluster analysis (HCA), principal component analysis (PCA), and artificial neural networks (ANN). Thereby, the degree of agreement between said sample hybridization (respectively binding) pattern and said reference hybridization (respectively binding) patterns can be determined by mathematical clustering methods.

Preferably, the degree of agreement between said sample hybridization (respectively binding) pattern and said reference hybridization (respectively binding) patterns is determined artificial neural networks.

The invention is explained and illustrated by the following example, without restriction of generality.

### EXAMPLE

This example is related to an analytical chip according to the invention for human diagnostics, capable for the simultaneous detection of bacterial 16S-rRNA from up to 50 different, clinically most relevant pathogenic bacteria for humans. Using sets of multiple immobilized, different polynucleotides for recognition of and hybridization with different subsequences of a 16S-rRNA out of the multitude of different 16S-rRNA derived from all 50 different bacteria, specific hybridization patterns can be established for each of them. The example is also related to an analytical method according to the invention, using said analytical chip, for assignment of an observed and recorded hybridization pattern to a certain bacterium.

I.

A) Analytical Chip

1. Evanescent Field Measurement Platform

An evanescent field measurement platform with the exterior dimensions of 57 mm width (in parallel to the grating lines of a grating structure (c) modulated in a layer (a) of the measurement platform)×14 mm length (perpendicular to the lines of the grating structure)×0.7 mm thickness is used. This evanescent field measurement platform can be combined with a plate of polycarbonate provided with recesses open towards said measurement platform and openings as fluid inlets towards its opposite side. Thereby the evanescent field measurement platform forms the inner bottom surface of an array of sample compartments, which are arranged (in this example) as a linear row of sample compartments with the interior dimensions of 5 mm width×7 mm length×0.15 mm height (above the evanescent field measurement platform). Combination of the polycarbonate plate with the evanescent field measurement platform as the base plate can, for example, be performed by gluing in such a way, that the recesses are tightly sealed against each other. Various embodiments of arrangements of sample compartments that can be generated using an analytical chip according to the invention are described in international patent applications WO 00/113096 and WO 00/143875, which are incorporated in this application in their full entirety. In this example, a linear arrangement of sample compartments is provided in such a form that can be inserted into a carrier (“meta carrier”) with the footprint of standard microtiter plates (about 85 mm×127 mm), the pitch of the inlets along one row of sample compartments being compatible with the pitch of the wells of a standard microtiter plate. In this preferred embodiment, the outlet of each sample compartment is fluidically connected to a reservoir as part of this sample compartment (flow cell) arrangement, for receiving liquid exiting the sample compartment. Thus, for example, washing steps can be performed without the need of emptying the sample compartments in between.

The evanescent field sensor platform as part of analytical chip according to the invention is provided as optical thin-film waveguide with a first optically transparent layer (a) in a second optically transparent layer (b) with lower refractive index than layer (a). The material of layer (a) (“substrate material”) consists of AF 45 glass (refractive index n=1.52 at 633 nm). A pair of surface relief gratings ((c), (c′)) is modulated in the substrate surface, on which during the further production process the second optically transparent and waveguiding layer (a) is deposited. Upon the deposition steps, the grating structures are reproduced in both the surface of layer (a) contacting layer (b) and into the opposite surface. The lines of the two grating structures (c), for coupling of excitation light into layer (a), and (c′), for coupling out light guided in layer (a), are oriented in parallel to the width of the evanescent field measurement platform, extending over the whole width. The grating period is 318 nm, the grating depth (12+/−3) nm. The distance between the two gratings is 9 mm, their length (in parallel to the length of the evanescent field measurement platform) 0.5 mm. The distance between the incoupling and the outcoupling grating of the pair of gratings is selected in such a way, that incoupling of excitation light can be performed within the base area of a sample compartment, formed by the combination of the evanescent field measurement platform with a polycarbonate plate as described above, whereas the outcoupling is performed outside of the sample compartments. The waveguiding, optically transparent layer (a) consists of Ta₂O₅ and has a thickness of 150 nm and a refractive index of 2.15 at 633 nm.

The sample compartments formed by the combination of the evanescent field measurement platform and the polycarbonate plate are provided with conical openings at the inner boundary surface opposite to the base plate, extending through the polycarbonate plate and thus allowing for filling or emptying the sample compartments upon inserting standard (micro) pipette tips.

As a preparation for the immobilization of the polynucleotides for specific recognition of and hybridization with bacterial 16S-rRNA, the evanescent field measurement platform is cleaned using organic and inorganic reagents (e.g. propanol and sulphuric acid, with intermediate steps of washing with water) upon ultra-sonication. These and all further processing steps including the immobilization of the polynucleotides or oligonucleotides are performed under clean room conditions. The cleaned evanescent field measurement platforms are dried and then stored under pollution-free conditions until further processing.

2. Deposition of an Adhesion-Promoting Layer

For improving the stability of the immobilized polynucleotides on the surface of the evanescent field measurement platform, an adhesion-promoting layer is deposited on the surface of said measurement platform: It is silanized with a functionalized silane in the liquid phase (2% v/v 3-Glycidyloxypropyl-trimethoxy silane in xylol). After washing and drying, the evanescent field measurement platforms are again stored under pollution-free conditions until further processing.

3. Capture Probe Selection and Array Design

Sequences of 16S-rRNA strands of selected bacteria can be derived, for example, from GenBank via direct access or various database aggregators. On average, five different 19-mer subsequences per bacterial 16S-rRNA were selected, for which complementary oligonucleotides were obtained as capture probes to be immobilized on the evanescent field measurement platform. The most important criteria for the capture probe selection were:
maximum diversity of the probe sequences within the full set of capture probes
optimum GC content
bias of positioning the probes towards the 3′ends of the 16S-rRNA strands
comparable melting points of the probe-target (16S-rRNA) complexes to be formed (ideally similarity of melting points within +/−1.5°)
selection of “stable” regions of the target 16S-rRNA, with rare occurrence of mutations, as subsequences to be probed, in order to avoid high molecular variability

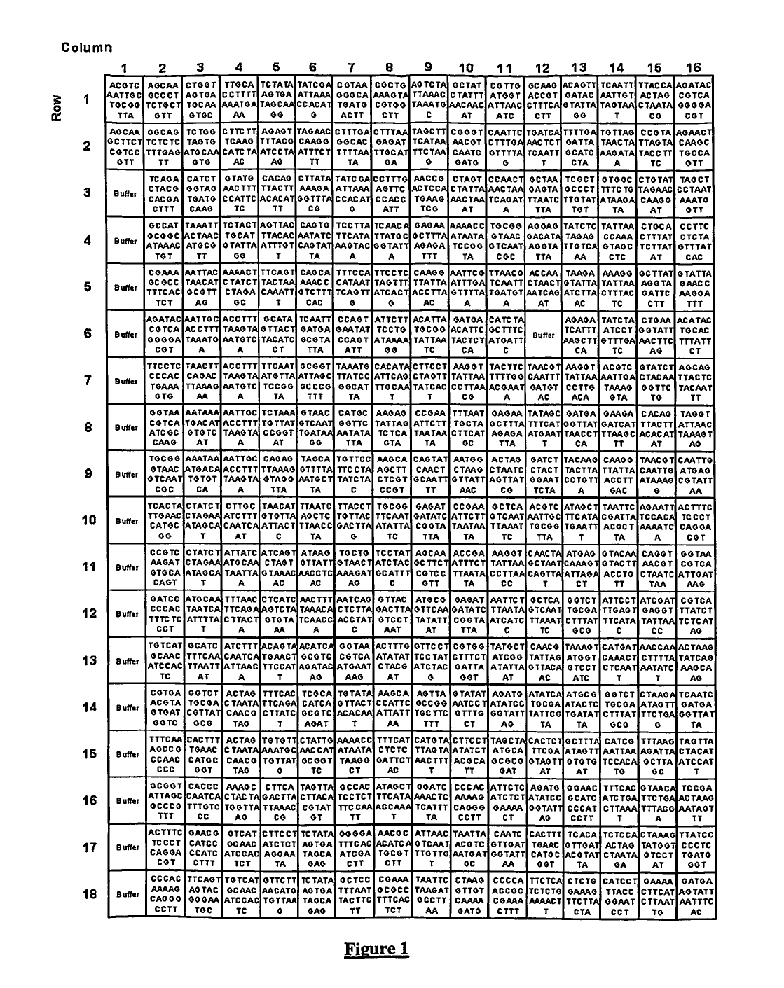

The design of an array capable for the determination of 21 different microorganisms using 272 different 19-mer capture probes (used for the correlation between hybridization patterns and identification of bacteria, example I.B.4), with 16 columns of measurement areas arranged in 18 rows, is depicted in FIG. 1. The related sequences are listed in Table 1.

4. Immobilization of Polynucleotides as Specific Recognition Elements

In this example, without restriction of generality, single-stranded 19-mer oligonucleotides, which have been functionalized with amino groups at their 5′ends, are used as recognition elements for specific recognition of and hybridization with essentially complementary 16S-rRNA to be detected in a supplied sample. The oligonucleotides are provided at a concentration of 50 μM in carbonate buffer (200 mM) and deposited on the silanized surface of the evanescent field measurement platform in discrete measurement areas using a commercial spotter (Virtek, Eurogentecs, Seraing, Belgium). Up to twelve hours are allowed for covalent binding of the recognition elements and drying of the surface.

B) Analytical Method

1. Sample Preparation

The sample preparation described here is used as a model system for samples to be taken and the material to be further processed from a whole blood sample in a real diagnostic application.

The bacteria to be determined are cultivated in a sugar-containing cultivation broth (in order to obtain enough material necessary for reference measurements using established methods requiring relatively large sample amounts). Then they are precipitated (“pelleted”) from the culture medium by centrifugation. The bacterial cell walls are disrupted. The whole contained RNA (“total RNA”) is subsequently isolated, using a commercial RNeasy Kit (Qiagen GmbH, Hilden, Germany).

The isolated “total RNA” is labeled with a rhodamine dye using a commercial “rhodamine nucleic acid labeling kit” (Kreatech Diagnostics, Amsterdam, The Netherlands). The kit comprises a platinum complex with two active binding sites (“Universal Linkage System”, ULS) for binding the fluorescence label (in this example the rhodamine dye) and the purin bases of the nucleic acids to be labeled.

This is an example of a “chemical” labeling method (by covalent binding), which is superior, in terms of reproducibility and labeling efficiency, to enzymatic labeling methods, for example by means of reverse transcriptase, as there is no dependence on enzymatic activity. Unbound fluorescence labels (respectively “ULS complexes) are removed upon processing with the RNeasy Kit, followed by a fragmentation step using T4 polynucleotide kinase that yields fragments typically in the range of 30-130 bases. The products were further purified using Centri-Sep columns (Princeton Separations, Adelphia, N.J., USA).

2. Hybridization with Rhodamine-ULS-Labeled Total RNA

The rhodamine-ULS-labeled total RNA, containing the (labeled) 16S-rRNA to be detected besides other polynucleotides, such as mRNA, tRNA, etc., is diluted with water and hybridization buffer “2×ZB1” (300 mM NaCl/30 mM sodium citrate, pH 7.5) in such a way that a final volume of 25 μl is obtained, at a buffer concentration of “1×ZB1” (150 mM NaCl, 15 mM sodium citrate, pH 7.5). The amounts of rhodamine-ULS-labeled total RNA, that are available for hybridization with the corresponding immobilized polynucleotides as capture probes of a total array of measurement areas, are between 2 ng and 500 ng. An amplification of the biological material (available total RNA) is not performed.

The prepared sample solution containing the labeled RNA is filled into a sample compartment housing an array of discrete measurement areas with 19-mer oligonucleotides immobilized on the silanized evanescent field measurement platform forming the bottom of the sample compartment. Hybridization of the immobilized capture probes with essentially complementary subsequences of 16S-rRNA contained in the sample is allowed for an incubation period of 60 minutes.

In order to increase the specificity of the hybridization, i.e., to minimize the amount of hybridization between sequences that are not completely complementary to each other, and especially to prevent unspecific hybridization, the hybridization step is performed under “stringent conditions”, for example at elevated temperature close to the melting temperature of the RNA to be detected (in this example at 50° C.).

For further increase of selectivity, the analytical chip with the formed hybrids is then washed under “increasingly stringent conditions” (temperature: 20° C.), first in washing buffer 1 (150 mM NaCl/15 mM sodium citrate, pH 7.5, with 0.1% SDS (sodium dodecyl sulfate)) for 5 minutes, then for 5 minutes in washing buffer 2 (15 mM NaCl/1.5 mM sodium citrate, pH 7.5, with 0.1% SDS), and finally for another 5 min in washing buffer 3 (15 mM NaCl/1.5 mM sidium citrate, pH 7.5), taking thereby advantage of the reservoirs integrated on the analytical chip according to the invention (see example I.A.1).

“Increasingly stringent condition” shall mean that the dissociation of hybrids formed between not completely complementary polynucleotide sequences (with one or more mismatches between the formed pairs) is enhanced at decreasing concentration of positively charged ions in the buffer solution, as well as with decreasing concentration of detergents, which thus results in an increase of the selectivity of the method.

After termination of the hybridization and the subsequent washing steps, the ensemble of a row of sample compartments formed by the analytical platform, carrying the hybridized labeled 16S-rRNA bound from the sample, and by the polycarbonate plate combined with it, is inserted into a “meta carrier” (see Example A.1) and then inserted into a ZeptoREADER™ for excitation and detection of luminescence signals emanating from the measurement areas, (see below, Example I.B.3.) especially resulting from the binding of luminescently labeled 16S-rRNA on the corresponding measurement areas, and for the laterally resolved detection of the background signal intensities. During this measurement, the sample compartments are filled with buffer washing buffer 2.

3. Analytical System and Measurement Method

Fluorescence signals from different complete arrays of measurement areas, arranged at a 9 mm pitch (compatible to the pitch of standard microtiter plates) are measured sequentially using a ZeptoREADER™ (Zeptosens AG, Witterswil, Switzerland). For measurement of each array of measurement areas, the analytical chip according to the invention is adjusted for fulfillment of the resonance condition for incoupling of light into the waveguiding tantalum pentoxide layer and for maximization of the excitation light available in the measurement areas.

Then a user-definable number of images of the fluorescence signals emanating from an array is generated for each array, wherein different exposure times can be chosen (typically in the range of 1 to 60 sec). The excitation wavelength for the measurements of the present example is 635 nm. Detection of the fluorescence light is performed using a cooled CCD camera at the emission wavelength of the fluorescence label upon using an interference filter (transmission (670+/−20) nm) positioned in front of the camera objective, for discrimination of scattered light at the excitation wavelength. The generated fluorescence images are automatically recorded on the hard disk of the control computer (for controlling the operation of the ZeptoREADER). Further details of the optical system (ZeptoREADER™) are described in the international patent application PCT/EP 01/10012, which is incorporated in this patent application in full entirety.

4. Data Analysis and Referencing

The medium signal intensity emanating from the measurement areas (spots) is determined using an image analysis software (ZeptoVIEW™, Zeptosens AG, CH-4108 Witterswil, Switzerland), which allows to analyze the fluorescence images of a multitude of arrays of measurement areas semi-automatically.

The raw data obtained from the individual pixels of the CCD (charge-coupled device) camera form a two-dimensional matrix of the digitized measurement data, with the measured intensity as the measurement value of a pixel corresponding to the surface section of the analytical chip imaged onto said pixel. For data analysis, at the beginning a two-dimensional (coordinate) net is superimposed over the image points (pixel values) in such a way that each measurement area (spot) is contained in an individual, two-dimensional net element. Within this net element, an “analysis element” (area of interest, “AOI”)) is assigned to each spot, with a geometry optimized for matching the spot geometry. These AOIs can have any geometric form, for example circular form. The location of the AOIs in the two-dimensional net is individually optimized as a function of the signal intensity recorded by the corresponding pixels. Dependent on the definitions set by the user, the initially defined radius of an AOI can be preserved or can be re-adjusted according to the geometry and size of a given spot. For example, the arithmetic average of the pixel values (signal intensities) can be determined as the mean gross signal intensity of every spot.

The background signals are determined from the signal intensities measured between the spots. For this purpose, for example, further circles can be defined, which are concentric with a given circular spot (and the assigned “spot AOI”), but have a larger radius. Of course, the radii of these concentric circles have to be smaller than the distance between adjacent spots. Then, for example, the region between the “spot AOI” and the first larger concentric circle can be disregarded, and the region between said first larger and a second still larger concentric circle can be defined as the AOI for the background determination (“background AOI”). It is also possible to define regions between adjacent spots, preferably located in the middle between adjacent spots, as AOIs for the determination of the background signal intensities. From these signal values the average background signal can then be determined in analogous way as described above, for example as the arithmetic average of the pixel values (signal intensities) of the chosen “background AOI”. The average net signal intensity can then be determined as the difference between the local average gross and the local average background signal intensity.

5. Correlation of Hybridization Patterns and Identification of Bacteria

The data sets derived from the analyzed images of the hybridization patterns characteristic for different 16S-rRNA applied on the analytical chip are stored on a computer hard-disk in a spread sheet format. For correlation with the appropriate genus and/or species information, the entirety of the resulting data sets (that can already be regarded as a data library) is analyzed using hierachical cluster analysis. If required, the information stored there can be reduced using principal component analysis and further analyzed e.g. by using learning artificial neural networks

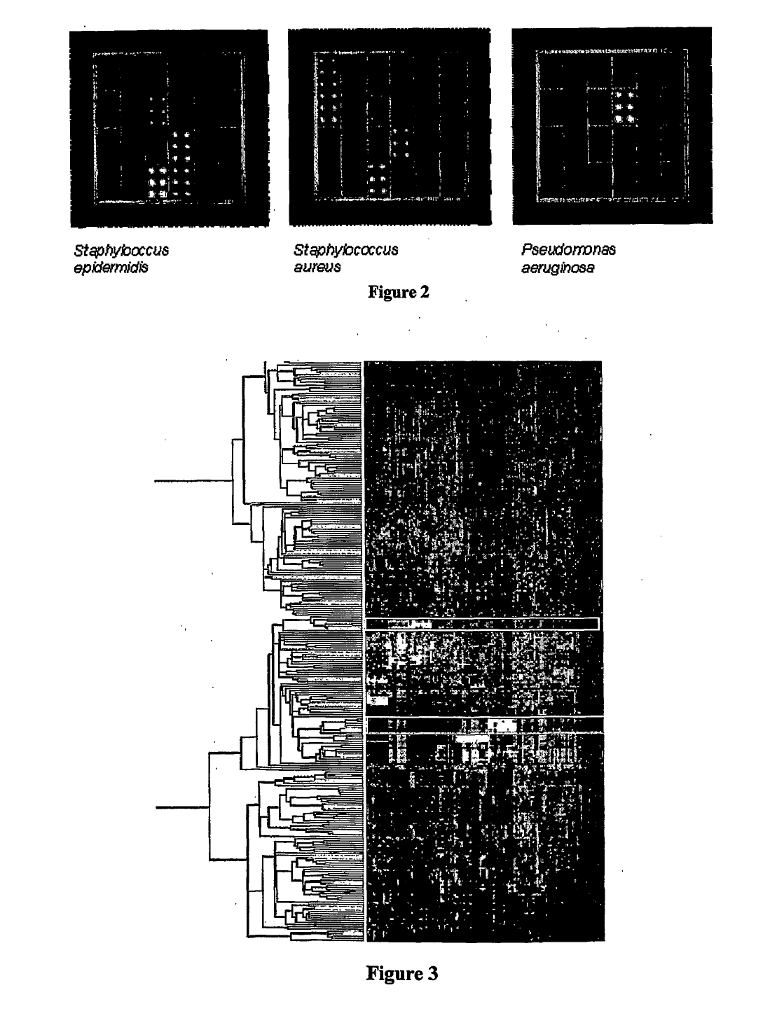

The hybridization patterns such as shown in FIG. 2 (from a smaller-sized array of 12×12 measurement areas) demonstrate the possibilities, dependent on the specifity of the target subsequences of 16S-rRNA to be detected by immobilized complementary oligonucleotides, to determine a common genus and to differentiate, between different geni (Staphylococcus, FIG. 2 left and center, versus Pseudomonas, FIG. 2, right), as well as to differentiate between different species of the same genus (Staphylococcus epidermidis, FIG. 2 left, versus Staphylococcus aureus, FIG. 2, center). With the array shown in this example, parts of the array showed a similar hybridization pattern for the two different staphylococcus species, whereas in other parts of the array (especially concerning the upper left corner of FIG. 2, left and center) considerable differences are observed.

FIG. 3 shows the full clustered pattern of data generated in 210 experiments, using the analytical chip described in Example I.A.3, for the determination of 21 different microorganisms using 272 different 19-mer capture probes. Clustering of the data was performed using the Average Linkage (UPGMA) variant of hierarchical cluster analysis. The y-axis shows the dendrogram of the clustered probes, the x-axis the different hybridization experiments ordered according to bacterium species and grouped for repetitive experiments.

The section enlargement of FIG. 4a shows—on the example of Pseudomonas aeruginosa (“Ps aerug”)—the strong correlation of probes selected for Peudomonas aeruginosa and the high signal intensity obtained in all experiments (light gray colors representing high signal intensities, in contrast to dark colors representing low signal intensities), where Pseudomonas aeroginosa 16S-rRNA is present, in contrast to experiments, e.g. where Enterococcus faecalis (“Efaecal”) or Streptococcus agalactiae (“Stagal”) are determined.

The section enlargement of FIG. 4b highlights the probes indicative for the bacterial genus Staphylococcus—shown on the example of hybridization with Staphylococcus aureus (“St aureu”) and Staphylococcus epidermidis (“St epide”), which leads to high signal intensities.

TABLE 1

| | | | | | |
|---|---|---|---|---|---|
| Running | Array | Array | | 19mer Probe | |
| Number | Column | Row | Short Name | Sequence | Complete name |

| | | | | | |
|---|---|---|---|---|---|
| 1 | 1 | 1 | Ecloac_411 | ACGTCAATTGCT | Enterobacter cloacae |
| | | | | GCGGTTA | |
| 2 | 1 | 2 | SA_15 | AGCAAGCTTCTC | Staphylococcus aureus |
| | | | | GTCCGTT | |
| 3 | 1 | 3 | Buffer | Buffer | Buffer |
| 4 | 1 | 4 | Buffer | Buffer | Buffer |
| 5 | 1 | 5 | Buffer | Buffer | Buffer |
| 6 | 1 | 6 | Buffer | Buffer | Buffer |
| 7 | 1 | 7 | Buffer | Buffer | Buffer |
| 8 | 1 | 8 | Buffer | Buffer | Buffer |
| 9 | 1 | 9 | Buffer | Buffer | Buffer |
| 10 | 1 | 10 | Buffer | Buffer | Buffer |
| 11 | 1 | 11 | Buffer | Buffer | Buffer |
| 12 | 1 | 12 | Buffer | Buffer | Buffer |
| 13 | 1 | 13 | Buffer | Buffer | Buffer |
| 14 | 1 | 14 | Buffer | Buffer | Buffer |
| 15 | 1 | 15 | Buffer | Buffer | Buffer |
| 16 | 1 | 16 | Buffer | Buffer | Buffer |
| 17 | 1 | 17 | Buffer | Buffer | Buffer |
| 18 | 1 | 18 | Buffer | Buffer | Buffer |
| 19 | 2 | 1 | Ecoli_70 | AGCAAGCCCTTC | Escherichia coli |
| | | | | TGCTGTT | |
| 20 | 2 | 2 | Ecoli_1093 | GGCAGTCTCTCTT | Escherichia coli |
| | | | | TGAGTT | |
| 21 | 2 | 3 | Ecoli_1251 | TCAGACTACGCA | Escherichia coli |
| | | | | CGACTTT | |
| 22 | 2 | 4 | Efaec_137 | GCCATGCGGCAT | Enterococcus faecalis |
| | | | | AAACTGT | |
| 23 | 2 | 5 | Efaec_159 | CGAAAGCGCCTT | Enterococcus faecalis |
| | | | | TCACTCT | |
| 24 | 2 | 6 | En_faeca_436 | AGATACCGTCAG | Enterococcus faecalis |
| | | | | GGGACGT | |
| 25 | 2 | 7 | Kpn_378 | TTCCTCCCCACTG | Klebsiella pneumoniae |
| | | | | AAAGTG | |
| 26 | 2 | 8 | Kpn_415 | GGTAACGTCAAT | Klebsiella pneumoniae |
| | | | | CGCCAAG | |
| 27 | 2 | 9 | Kpn_419 | TGCGGGTAACGT | Klebsiella pneumoniae |
| | | | | CAATCGC | |
| 28 | 2 | 10 | St_epide_137 | TCACTATTGAAC | Not_Staph aureus |
| | | | | CATGCGG | |
| 29 | 2 | 11 | St_aureu_420 | CCGTCAAGATGT | Not_Staph epidermidis |
| | | | | GCACAGT | |
| 30 | 2 | 12 | Psae_129 | GATCCCCCACTTT | Pseudo aeruginosa |
| | | | | CTCCCT | |
| 31 | 2 | 13 | StPn_126 | TGTCATGCAACA | Streptococcus |
| | | | | TCCACTC | pneumoniae |
| 32 | 2 | 14 | StPn_159 | CGTGAACGTAGT | Streptococcus |
| | | | | GATGGTC | pneumoniae |
| 33 | 2 | 15 | Pac_153 | TTTCAAAGCCGC | Propionibacterium |
| | | | | CAACCCC | acnes |
| 34 | 2 | 16 | Psae_100 | GCGGTATTAGCG | Pseudo aeruginosa |
| | | | | CCCGTTT | |
| 35 | 2 | 17 | Psae_121 | ACTTTCTCCCTCA | Pseudo aeruginosa |
| | | | | GGACGT | |
| 36 | 2 | 18 | Pac_17 | CCCACAAAAGCA | Propionibacterium |
| | | | | GGGCCTT | acnes |
| 37 | 3 | 1 | St_pneum_159 | CTGGTAGTGATG | Streptococcus |
| | | | | CAAGTGC | pneumoniae |
| 38 | 3 | 2 | St_pneum_160 | TCTGGTAGTGAT | Streptococcus |
| | | | | GCAAGTG | pneumoniae |
| 39 | 3 | 3 | St_pneum_162 | CATCTGGTAGTG | Streptococcus |
| | | | | ATGCAAG | pneumoniae |
| 40 | 3 | 4 | St_pyoge_136 | TAAATTACTAAC | Streptococcus |
| | | | | ATGCGTT | pyogenes |
| 41 | 3 | 5 | St_pyoge_134 | AATTACTAACAT | Streptococcus |
| | | | | GCGTTAG | pyogenes |
| 42 | 3 | 6 | St_pyoge_193 | AATTGCACCTTTT | Streptococcus |
| | | | | AAATGA | pyogenic consensus |
| 43 | 3 | 7 | St_pyoge_590 | TAACTTCAGACTT | Streptococcus |
| | | | | AAAGAA | pyogenic consensus |
| 44 | 3 | 8 | St_saliv_134 | AATAAATGACAT | Streptococcus |
| | | | | GTGTCAT | salivarius |
| 45 | 3 | 9 | St_saliv_135 | AAATAAATGACA | Streptococcus |
| | | | | TGTGTCA | salivarius |
| 46 | 3 | 10 | St_saliv_910 | CTATCTCTAGAA | Streptococcus |
| | | | | ATAGCAT | salivarius consensus |
| 47 | 3 | 11 | St_saliv_910 | CTATCTCTAGAA | Streptococcus |
| | | | | ATAGCAT | salivarius consensus |
| 48 | 3 | 12 | St_sangu_124 | ATGCAATAATCA | Streptococcus sanguis |
| | | | | ATTTTAT | |
| 49 | 3 | 13 | St_sangu_144 | GCATCTTTCAATT | Streptococcus sanguis |
| | | | | AATTAT | |
| 50 | 3 | 14 | Ecloac_120 | GGTCTTGCGACG | Enterobacter cloac |
| | | | (G) | TTATGCG | |
| 51 | 3 | 15 | All_Staph_175 | CACTTTTGAACC | Staph consencus |
| | | | | ATGCGGT | |
| 52 | 3 | 16 | All_Staph_941 | CACCCCAATCAT | Staph consensus |
| | | | | TTGTCCC | |
| 53 | 3 | 17 | Bfrag_173 | GAACGCATCCCC | Bacteroides fragilis |
| | | | | ATCCTTT | |
| 54 | 3 | 18 | Eaggl_1131 | TTCAGTAGTACG | Entero agglomerans |
| | | | | GGAATGC | |
| 55 | 4 | 1 | St_dysga_146 | TTGCACCTTTTAA | Streptococcus |
| | | | | ATGAAA | dysgalactiae |
| 56 | 4 | 2 | St_equin_143 | CTTCTTTCAAGCA | Streptococcus equinus |
| | | | | TCTAAC | |
| 57 | 4 | 3 | St_inter_420 | GTATGAACTTTCC | Streptococcus |
| | | | | ATTCTC | intermedius |
| 58 | 4 | 4 | St_miti_40 | TCTACTTGCATGT | Streptococcus mitis |
| | | | | ATTAGG | |
| 59 | 4 | 5 | St_miti_1006 | AAAACTCTATCT | Streptococcus mitis |
| | | | | CTAGAGC | |
| 60 | 4 | 6 | St_mitis_187 | ACCTTTTAAGTA | Streptococcus mitis |
| | | | | AATGTCA | consensus |
| 61 | 4 | 7 | St_mitis_187 | ACCTTTTAAGTA | Streptococcus mitis |
| | | | | AATGTCA | consensus |
| 62 | 4 | 8 | St_mitis_193 | AATTGCACCTTTT | Streptococcus mitis |
| | | | | AAGTAA | consensus |
| 63 | 4 | 9 | St_mitis_193 | AATTGCACCTTTT | Streptococcus mitis |
| | | | | AAGTAA | consensus |
| 64 | 4 | 10 | St_mutan_155 | CTTGCATCTTTCA | Streptococcus mutans |
| | | | | ATCAAT | |
| 65 | 4 | 11 | St_mutan_138 | ATTATCATGCAA | Streptococcus mutans |
| | | | | TAATTAA | |
| 66 | 4 | 12 | St_mutan_529 | TTTAACTTCAGAC | Streptococcus mutans |
| | | | | TTACTA | consensus |
| 67 | 4 | 13 | St_mutan_127 | ATCTTTCAATCAA | Streptococcus mutans |
| | | | | TTAACA | consensus |
| 68 | 4 | 14 | St_pneum_136 | ACTAGCTAATAC | Streptococcus |
| | | | | AACGTAG | pneumonia |
| 69 | 4 | 15 | St_pneum_136 | ACTAGCTAATAC | Streptococcus |
| | | | | AACGTAG | pneumonia |
| 70 | 4 | 16 | St_pneum_585 | AAAGCCTACTAT | Streptococcus |
| | | | | GGTTAAG | pneumonia consensus |
| 71 | 4 | 17 | St_pneum_125 | GTCATGCAACAT | Streptococcus |
| | | | | CCACTCT | pneumoniae |
| 72 | 4 | 18 | St_pneum_126 | TGTCATGCAACA | Streptococcus |
| | | | | TCCACTC | pneumoniae |
| 73 | 5 | 1 | St_kloos_163 | TCTATAAGTGAT | Staphylococcus kloosii |
| | | | | AGCAAGG | |
| 74 | 5 | 2 | St_lugdu_372 | AGAGTTTTACGA | Staphylococcus |
| | | | | TCCTAAG | lugdunensis |
| 75 | 5 | 3 | St_sapro_407 | CACAGTTACTTA | Staphylococcus |
| | | | | CACATTTT | saprophyticu |
| 76 | 5 | 4 | St_sapro_404 | AGTTACTTACAC | Staphylococcus |
| | | | | ATTTGTT | saprophyticu |
| 77 | 5 | 5 | St_sciur_408 | TTCAGTTACTAAC | Staphylococcus sciuri |
| | | | | AAATTT | |
| 78 | 5 | 6 | St_simul_408 | GCATAGTTACTT | Staphylococcus |
| | | | | ACATCCT | simulans |
| 79 | 5 | 7 | St_warne_118 | TTCAATATGTTAT | Staphylococcus |
| | | | | CCGGTA | warneri |
| 80 | 5 | 8 | St_xylos_117 | TCTAAATGTTATC | Staphylococcus |
| | | | | CGGTAT | xylosus |
| 81 | 5 | 9 | St_mucil_69 | CAGAGTTAAAGG | Stomatococcus |
| | | | | TAGGTTA | mucilaginosus |
| 82 | 5 | 10 | St_agala_128 | TAACATGTGTTTA | Streptococcus |
| | | | | ATTACTC | agalactiae |
| 83 | 5 | 11 | St_agala_25 | ATCAGTCTAGTG | Streptococcus |
| | | | | TAAACAC | agalactiae |
| 84 | 5 | 12 | St_agala_28 | CTCATCAGTCTA | Streptococcus |
| | | | | GTGTAAA | agalactiae |
| 85 | 5 | 13 | St_angin_365 | ACAGTATGAACT | Streptococcus |
| | | | | TTCCATT | anginosus consensus |
| 86 | 5 | 14 | St_angin_494 | TTTCACTTCAGAC | Streptococcus |
| | | | | TTATCT | anginosus consensus |
| 87 | 5 | 15 | St_bovis_159 | TGTGTTAAATGCT | Streptococcus bovis |
| | | | | GTTATG | consensus |
| 88 | 5 | 16 | St_bovis_575 | CTTCAGACTTATT | Streptococcus bovis |
| | | | | AAACCG | consensus |
| 89 | 5 | 17 | St_bovis_995 | CTTCCTATCTCTA | Streptococcus bovis |
| | | | | GGAATA | consensus |
| 90 | 5 | 18 | St_canis_133 | GTTCTTAACATGT | Streptococcus canis |
| | | | | GTTAAG | |
| 91 | 6 | 1 | Ci_freun_949 | TATCGAATTAAA | Citrobacter freundi |
| | | | | CCACATG | |
| 92 | 6 | 2 | Bu_cepac_389 | TAGAACCAAGGA | Burkholderia cepacia |
| | | | | TTTCTTT | |
| 93 | 6 | 3 | Ba_dista_377 | CTTATAAAAGAG | Bacteroides distasonis |
| | | | | GTTTACG | |
| 94 | 6 | 4 | Ac_meyer_111 | CAGTGAATATCC | Actinomyces meyeri |
| | | | | AGTATTA | |
| 95 | 6 | 5 | St_epide_152 | CAGCAAAACCGT | Staphylococcus |
| | | | | CTTTCAC | epidermidis |
| 96 | 6 | 6 | Se_marce_465 | TCAATTGATGAG | Serratia marcescen |
| | | | | CGTATTA | |
| 97 | 6 | 7 | Ps_aerug_100 | GCGGTATTAGCG | Pseudomonas |
| | | | | CCCGTTT | aeruginosa |
| 98 | 6 | 8 | Pr_mirab_416 | GTAACGTCAATT | Proteus mirabilis |
| | | | | GATAAGG | |
| 99 | 6 | 9 | Pe_conse_617 | TAGCAGTTTTAA | PeptoStreptococcus |
| | | | | ATGCTTA | consensus |
| 100 | 6 | 10 | Le_pneum_405 | TTAATCAGCTCTT | Legionella |
| | | | | AACCTA | pneumophila |
| 101 | 6 | 11 | Kl_oxyto_401 | ATAAGGTTATTA | Klebsiella oxytoca |
| | | | | ACCTCAC | |
| 102 | 6 | 12 | Ge_haemo_144 | AACTTTTAAACA | Gemella haemolysans |
| | | | | TCAACCA | |
| 103 | 6 | 13 | St_epide_210 | ACATCAGCGTCA | Staphylococcus |
| | | | | GATACAG | epidermidis |
| 104 | 6 | 14 | St_epide_214 | TCGCACATCAGC | Staphylococcus |
| | | | | GTCAGAT | epidermidis |
| 105 | 6 | 15 | St_epide_134 | CTATTGAACCAT | Staphylococcus |
| | | | | GCGGTTC | epidermidis |
| 106 | 6 | 16 | St_haemo_405 | TAGTTACTTACAC | Staphylococcus |
| | | | | GTATGT | haemolyticus |
| 107 | 6 | 17 | St_homin_163 | TCTATAAGTGAT | Staphylococcus |
| | | | | AGCAGAG | hominis |
| 108 | 6 | 18 | St_homin_163 | TCTATAAGTGAT | Staphylococcus |
| | | | | AGCAGAG | hominis |
| 109 | 7 | 1 | En_aggl._1131 | CGTAAGGGCATG | Enterobacter aggl. |
| | | | | ATGACTT | |
| 110 | 7 | 2 | Cl_ramos_137 | CTTTGAGGCACTT | Clostridium ramosum |
| | | | | TTAATA | |
| 111 | 7 | 3 | Ci_freun_949 | TATCGAATTAAA | Citrobacter freundi |
| | | | | CCACATG | |
| 112 | 7 | 4 | Ba_vulga_430 | TCCTTATTCATAA | Bacteroides vulgatus |
| | | | | AGTACA | |
| 113 | 7 | 5 | Ba_conse_1012 | TTTCCACATAATT | Bacteroides consensus |
| | | | | CAGTTG | |
| 114 | 7 | 6 | Ac_meyer_112 | CCAGTGAATATC | Actinomyces meyeri |
| | | | | CAGTATT | |
| 115 | 7 | 7 | St_cohni_115 | TAAATGTTATCC | Staphylococcus cohnii |
| | | | | GGCATTA | |
| 116 | 7 | 8 | St_epide_64 | CATGCGGTTCAA | Staphylococcus |
| | | | | TATATTA | epidermidis |
| 117 | 7 | 9 | Ps_aerug_11 | TGTTCCTTCCTAT | Pseudomonas |
| | | | | ATCTAC | aeruginos |
| 118 | 7 | 10 | Pr_conse_1476 | TTACCTTGTTACG | Propionibacterium |
| | | | | ACTTAG | consensus |
| 119 | 7 | 11 | Pe_conse_1114 | TGCTGGTAACTA | PeptoStreptococcus |
| | | | | AAGATAG | consensus |
| 120 | 7 | 12 | Le_pneum_403 | AATCAGCTCTTA | Legionella |
| | | | | ACCTATC | pneumophila |
| 121 | 7 | 13 | Kl_pneum_415 | GGTAACGTCAAT | Klebsiella pneumoniae |
| | | | | GAATAAG | |
| 122 | 7 | 14 | Ge_haemo_410 | TGTATAGTTACTA | Gemella haemolysans |
| | | | | CACAAT | |
| 123 | 7 | 15 | Eu_limos_130 | AAAACCATAATA | Eubacterium limosum |
| | | | | TAAGGCT | |
| 124 | 7 | 16 | En_faeca_42 | GCCACTCCTCTTT | Enterococcus faecalis |
| | | | | CCAATT | |
| 125 | 7 | 17 | En_aggl._533 | GGGGATTTCACA | Enterobacter aggl. |
| | | | | TCGACTT | |
| 126 | 7 | 18 | Cl_terti_141 | GCTCCTTTAATTA | Clostridium tertium |
| | | | | CTTCTT | |
| 127 | 8 | 1 | Es_coli_475 | CGCTGAAAGTAC | Escherichia coli |
| | | | | GTGGCTT | |
| 128 | 8 | 2 | En_faeca_1261 | CTTTAAGAGATTT | Enterococcus faecali |
| | | | | GCATGA | |
| 129 | 8 | 3 | En_aggl._1071 | CCTTTGAGTTCCC | Enterobacter aggl. |
| | | | | ACCATT | |
| 130 | 8 | 4 | Cl_perfr_111 | TCAACATTATGC | Clostridium |
| | | | | GGTATTA | perfringens |
| 131 | 8 | 5 | Ci_freun_1163 | TTCCTCTAGTTTA | Citrobacter freundi |
| | | | | TCACTG | |
| 132 | 8 | 6 | Ba_ureol_378 | ATTCTTTCCTGAT | Bacteroides |
| | | | | AAAAGG | ureolyticus |
| 133 | 8 | 7 | Ba_conse_1008 | CACATAATTCAG | Bacteroides consensus |
| | | | | TTGCAAT | |
| 134 | 8 | 8 | Ac_junii_395 | AAGAGTATTAGT | Acinetobacter junii |
| | | | | CTCAGTA | |
| 135 | 8 | 9 | St_aureu_16 | AAGCAAGCTTCT | Staphylococcus aureus |
| | | | | CGTCCGT | |
| 136 | 8 | 10 | St_epide_62 | TGCGGTTCAATA | Staphylococcus |
| | | | | TATTATC | epidermidis |
| 137 | 8 | 11 | Ps_aerug_4 | TCCTATATCTACG | Pseudomonas |
| | | | | CATTTC | aeruginos |
| 138 | 8 | 12 | Pr_conse_1469 | GTTACGACTTAG | Propionibacterium |
| | | | | TCCTAAT | consensus |
| 139 | 8 | 13 | Pe_anaer_145 | ACTTTGATATATC | PeptoStreptococcus |
| | | | | TACGAT | anaerobi |
| 140 | 8 | 14 | La_conse_85 | AAGCACCATTCA | Lactobacillus |
| | | | | TTATTAA | consensus |
| 141 | 8 | 15 | Ha_parap_25 | TTTCATCTCTCGA | Haemophilus |
| | | | | TTCTAC | paraphrophilus |
| 142 | 8 | 16 | Fu_variu_130 | ATAGCTTTCATA | Fusobacterium varium |
| | | | | ACCAAAT | |
| 143 | 8 | 17 | Es_coli_899 | AACGCACATCAT | Escherichia coli |
| | | | | GCGTCTT | |
| 144 | 8 | 18 | En_faeca_159 | CGAAAGCGCCTT | Enterococcus faecalis |
| | | | | TCACTCT | |
| 145 | 9 | 1 | Ha_parai_400 | AGTCTATTAAAC | Haemophilus |
| | | | | TAAATGC | parainfluenzae |
| 146 | 9 | 2 | Fu_nucle_140 | TAGCTTTCATAAT | Fusobacterium |
| | | | | TCTAAG | nucleatum |
| 147 | 9 | 3 | Es_coli_1283 | AACCGACTCCAT | Escherichia coli |
| | | | | GAAGTCG | |
| 148 | 9 | 4 | En_faeca_1267 | GAGAAGCTTTAA | Enterococcus faecali |
| | | | | GAGATTT | |
| 149 | 9 | 5 | En_aerog_400 | CAAGGTTATTAA | Enterobacter aerogenes |
| | | | | CCTTAAC | |
| 150 | 9 | 6 | Cl_perfr_108 | ACATTATGCGGT | Clostridium |
| | | | | ATTAATC | perfringens |
| 151 | 9 | 7 | Ci_freun_1164 | CTTCCTCTAGTTT | Citrobacter freundi |
| | | | | ATCACT | |
| 152 | 9 | 8 | Ba_theta_155 | CCGAAATTCTTTA | Bacteroides |
| | | | | ATAATA | thetaiotaomicro |
| 153 | 9 | 9 | Ba_conse_639 | CAGTATCAACTG | Bacteroides consensus |
| | | | | CAATTTT | |
| 154 | 9 | 10 | Ac_conse_167 | GAGATGATATCC | Acinetobacter |
| | | | | GGTATTA | consensus |
| 155 | 9 | 11 | Es_coli_70 | AGCAAGCTTCTC | Escherichia coli |
| | | | | GTCCGTT | |
| 156 | 9 | 12 | St_epide_63 | ATGCGGTTCAAT | Staphylococcus |
| | | | | ATATTAT | epidermidis |
| 157 | 9 | 13 | Ps_aerug_10 | GTTCCTTCCTATA | Pseudomonas |
| | | | | TCTACG | aeruginos |
| 158 | 9 | 14 | Pr_acnes_418 | AGTTAGCCGGTG | Propionibacterium |
| | | | | CTTCTTT | acnes |
| 159 | 9 | 15 | Pe_anaer_108 | CATGTATTAGTA | PeptoStreptococcus |
| | | | | AACTTTT | anaerobi |
| 160 | 9 | 16 | La_conse_7 | GGATCAAACTCT | Lactobacillus |
| | | | | CATTTTA | consensus |
| 161 | 9 | 17 | Ha_parap_417 | ATTAACGTCAAT | Haemophilus |
| | | | | TTGTTGT | paraphrophilus |
| 162 | 9 | 18 | Fu_nucle_131 | TAATTCTAAGAT | Fusobacterium |
| | | | | GCCTTAA | nucleatum |
| 163 | 10 | 1 | Pa_multo_404 | GCTATCTATTTAA | Pasteurella multocida |
| | | | | CAACAT | |
| 164 | 10 | 2 | Kl_pneum_444 | CGGGTAACGTCA | Klebsiella pneumoniae |
| | | | | ATCGATG | |
| 165 | 10 | 3 | Ha_parai_402 | CTAGTCTATTAA | Haemophilus |
| | | | | ACTAAAT | parainfluenzae |
| 166 | 10 | 4 | Fu_necro_103 | AAAACCATAATA | Fusobacterium |
| | | | | TCCGGTA | necrophorum |
| 167 | 10 | 5 | Es_col_865 | AATTCGATTTGA | Escherichiacol |
| | | | | GTTTTAA | |
| 168 | 10 | 6 | En_conse_450 | GATGAACATTCT | Enterococcus |
| | | | | ACTCTCA | consensus |
| 169 | 10 | 7 | En_aerog_399 | AAGGTTATTAAC | Enterobacter aerogenes |
| | | | | CTTAACG | |
| 170 | 10 | 8 | Cl_parap_134 | TTTAATTGCTACT | Clostridium |
| | | | | TCATGC | paraputrificum |
| 171 | 10 | 9 | Ci_amalo_408 | AATGGCTAAGGT | Citrobacter |
| | | | | TATTAAC | amalonaticus |
| 172 | 10 | 10 | Ba_theta_155 | CCGAAATTCTTTA | Bacteroides |
| | | | | ATAATA | thetaiotaomicro |
| 173 | 10 | 11 | Ba_cacca_156 | ACCGAATTTCTTT | Bacteroides caccae |
| | | | | AATATA | |
| 174 | 10 | 12 | Ac_conse_167 | GAGATGATATCC | Acinetobacter |
| | | | | GGTATTA | consensus |
| 175 | 10 | 13 | St_aureu_440 | CGTGGCTTTCTGA | Staphylococcus aureus |
| | | | | TTAGGT | |
| 176 | 10 | 14 | Sa_enter_776 | GTATATAATCCT | Salmonella enteritidi |
| | | | | GTTTGCT | |
| 177 | 10 | 15 | Ps_aerug_6 | CTTCCTATATCTA | Pseudomonas |
| | | | | CGCATT | aeruginos |
| 178 | 10 | 16 | Pr_acnes_17 | CCCACAAAAGCA | Propionibacterium |
| | | | | GGGCCTT | acnes |
| 179 | 10 | 17 | Pa_multo_421 | TAATTAACGTCA | Pasteurella multocida |
| | | | | ATGATGC | |
| 180 | 10 | 18 | La_conse_1061 | CTAAGGTTGTCA | Lactobacillus |
| | | | | AAAGATG | consensus |
| 181 | 11 | 1 | Pr_stuar_403 | CGTTGATGGTATT | Providencia stuartii |
| | | | | AACATC | |
| 182 | 11 | 2 | Pr_acne_877 | CAATTCCTTTGAG | Propionibacterium |
| | | | | TTTTAG | acne |
| 183 | 11 | 3 | Ne_flave_183 | CCAACTAACTAA | Neisseria flavescens |
| | | | | TCAGATA | |
| 184 | 11 | 4 | Kl_pneum_419 | TGCGGGTAACGT | Klebsiella pneumoniae |
| | | | | CAATCGC | |
| 185 | 11 | 5 | Ha_influ_415 | TTAACGTCAATTT | Haemophilus |
| | | | | GATGTA | influenzae |
| 186 | 11 | 6 | Fu_necro_133 | CATCTAGCTTTCA | Fusobacterium |
| | | | | TGATTC | necrophorum |
| 187 | 11 | 7 | Es_col_1404 | TACTTCTTTTGGA | Escherichiacol |
| | | | | CGAATA | |
| 188 | 11 | 8 | En_conse_1271 | GAGAAGCTTTAA | Enterococcus |
| | | | | GAGATTA | consensus |
| 189 | 11 | 9 | Ei_corro_181 | ACTAGCTAATCA | Eikenella corrodens |
| | | | | GTTATCG | |
| 190 | 11 | 10 | Cl_innoc_42 | GCTCAGTCAATTT | Clostridium innocuum |
| | | | | AAATTC | |
| 191 | 11 | 11 | Ci_amalo_401 | AAGGTTATTAAC | Citrobacter |
| | | | | CTTAACC | amalonaticus |
| 192 | 11 | 12 | Ba_sterc_150 | AATTCTTTAATAA | Bacteroides stercoris |
| | | | | TCATCC | |
| 193 | 11 | 13 | Ba_cacca_119 | TATGCTATCGGA | Bacteroides caccae |
| | | | | TATTAAT | |
| 194 | 11 | 14 | Ac_conse_166 | AGATGATATCCG | Acinetobacter |
| | | | | GTATTAG | consensus |
| 195 | 11 | 15 | St_aureu_185 | TAGCTAATGCAG | Staphylococcus aureus |
| | | | | CGCGGAT | |
| 196 | 11 | 16 | Sa_conse_1014 | ATTCTCATCTCTG | Salmonella consensus |
| | | | | AAAACT | |
| 197 | 11 | 17 | Pr_stuar_407 | CAATCGTTGATG | Providencia stuartii |
| | | | | GTATTAA | |
| 198 | 11 | 18 | Pr_acnes_153 | CCCCAACCGCCG | Propionibacterium |
| | | | | AAACTTT | acnes |
| 199 | 12 | 1 | St_aureu_150 | GCAAGACCGTCT | Staphylococcus aureus |
| | | | | TTCACTT | |
| 200 | 12 | 2 | Sa_conse_1 | TGATCAAACTCTT | Salmonella consensus |
| | | | | CAATTT | |
| 201 | 12 | 3 | Pr_vulga_404 | GCTAAGAGTATT | Proteus vulgaris |
| | | | | AATCTTA | |
| 202 | 12 | 4 | Ae_virid_407 | AGGAGGACATAA | Aerococcus viridans |
| | | | | GGTATTA | |
| 203 | 12 | 5 | Ne_flave_184 | ACCAACTAACTA | Neisseria flavescens |
| | | | | ATCAGAT | |
| 204 | 12 | 6 | Empty | Buffer | EMPTY |
| 205 | 12 | 7 | Ha_influ_414 | TAACGTCAATTT | Haemophilus |
| | | | | GATGTAC | influenzae |
| 206 | 12 | 8 | Fu_morti_131 | TATAGCTTTCATA | Fusobacterium |
| | | | | TGAATT | mortiferum |
| 207 | 12 | 9 | Es_col_672 | GATCTCTACTGG | Escherichiacol |
| | | | | AATTCTA | |
| 208 | 12 | 10 | En_cloac_411 | ACGTCAATTGCT | Enterobacter cloacae |
| | | | | GCGGTTA | |
| 209 | 12 | 11 | Ei_corro_183 | CAACTAGCTAAT | Eikenella corrodens |
| | | | | CAGTTAT | |
| 210 | 12 | 12 | Cl_innoc_42 | GCTCAGTCAATTT | Clostridium innocuum |
| | | | | AAATTC | |
| 211 | 12 | 13 | Ch_luteo_395 | CAACGTATTAGG | Chryseomonas luteola |
| | | | | TTACAAC | |
| 212 | 12 | 14 | Ba_ovatu_136 | ATATCATGCGAT | Bacteroides ovatus |
| | | | | ATTCGTA | |
| 213 | 12 | 15 | Al_faeca_95 | CACTCTTTCGAGT | Alcaligenes faecalis |
| | | | | AGTTAT | |
| 214 | 12 | 16 | Ac_conse_166 | AGATGATATCCG | Acinetobacter |
| | | | | GTATTAG | consensus |
| 215 | 12 | 17 | St_aureu_175 | CACTTTTGAACC | Staphylococcus aureus |
| | | | | ATGCGGT | |
| 216 | 12 | 18 | Sa_conse_1013 | TTCTCATCTCTGA | Salmonella consensus |
| | | | | AAACTT | |
| 217 | 13 | 1 | Ae_hydro_410 | ACAGTTGATACG | Aeromonas hydrophila |
| | | | | TATTAGG | |
| 218 | 13 | 2 | Ac_conse_1252 | TTTTGAGATTAGC | Acinetobacter |
| | | | | ATCCTA | consensus |
| 219 | 13 | 3 | St_aureu_1237 | TCGCTGCCCTTTG | Staphylococcus aureus |
| | | | | TATTGT | |
| 220 | 13 | 4 | St_aureu_994 | TATCTCTAGAGTT | Staphylococcus aureus |
| | | | | GTCAAA | |
| 221 | 13 | 5 | Pr_vulga_402 | TAAGAGTATTAA | Proteus vulgaris |
| | | | | TCTTAAC | |
| 222 | 13 | 6 | Pe_prevo_16 | AGAGATCATTTA | PeptoStreptococcus |
| | | | | AGCTTCA | prevotii |
| 223 | 13 | 7 | Mo_morga_401 | AAGGTTATTAAC | Morganella morganii |
| | | | | CTTGACA | |
| 224 | 13 | 8 | Kl_pneum_429 | GATGAGGTTATT | Klebsiella pneumonia |
| | | | | AACCTCA | |
| 225 | 13 | 9 | Ha_aphro_16 | TACAAGTACTTA | Haemophilus |
| | | | | CCTGTTA | aphrophilus |
| 226 | 13 | 10 | Fu_morti_130 | ATAGCTTTCATAT | Fusobacterium |
| | | | | GAATTT | mortiferum |
| 227 | 13 | 11 | Es_col_436 | ATGAGCAAAGTA | Escherichiacol |
| | | | | TTAGACT | |
| 228 | 13 | 12 | En_cloac_120 | GGTCTTGCGACTT | Enterobacter cloacae |
| | | | | TATGCG | |
| 229 | 13 | 13 | Co_conse_176 | TAAAGTATGGTG | Corynebacterium |
| | | | | TCCTATC | consensus |
| 230 | 13 | 14 | Cl_conse_200 | ATGCGATACTCT | Clostridium consensus |
| | | | | GATATTA | |
| 231 | 13 | 15 | Ca_ochra_136 | GCTTTAATAGTTG | Capnocytophaga |
| | | | | TGTGAT | ochracea |
| 232 | 13 | 16 | Ba_fragi_174 | GGAACGCATCCC | Bacteroides fragilis |
| | | | | CATCCTT | |
| 233 | 13 | 17 | Ae_hydro_412 | TCACAGTTGATA | Aeromonas hydrophila |
| | | | | CGTATTA | |
| 234 | 13 | 18 | Ac_conse_978 | CTCTGGAAAGTT | Acinetobacter |
| | | | | CTTACTA | consensus |
| 235 | 14 | 1 | Ca_conse_718 | TCAATTAATTGTT | Capnocytophaga |
| | | | | AGTAAT | consensus |
| 236 | 14 | 2 | Ba_fragi_1112 | TGTTAGTAACTA | Bacteroides fragili |
| | | | | AAGATAA | |
| 237 | 14 | 3 | Ae_virid_439 | GTGGCTTTCTGAT | Aerococcus viridans |
| | | | | AAGATA | |
| 238 | 14 | 4 | Ac_conse_428 | TATTAACCAAAG | Acinetobacter |
| | | | | TAGCCTC | consensus |
| 239 | 14 | 5 | Sh_dysen_435 | AAAGGTATTAAC | Shigella dysenteria |
| | | | | TTTACTC | |
| 240 | 14 | 6 | St_aureu_768 | TATCTAATCCTGT | Staphylococcus aureus |
| | | | | TTGATC | |
| 241 | 14 | 7 | Pr_penne_413 | ACGTCAATTGAT | Proteus penneri |
| | | | | AAAGGTA | |
| 242 | 14 | 8 | Pe_prevo_18 | GAAGAGATCATT | PeptoStreptococcus |
| | | | | TAAGCTT | prevotii |
| 243 | 14 | 9 | Mo_morga_402 | CAAGGTTATTAA | Morganella morganii |
| | | | | CCTTGAC | |
| 244 | 14 | 10 | Kl_pneum_516 | TAATTCCGATTA | Klebsiella pneumonia |
| | | | | ACGCTTA | |
| 245 | 14 | 11 | Ha_aphro_17 | GTACAAGTACTT | Haemophilus |
| | | | | ACCTGTT | aphrophilus |
| 246 | 14 | 12 | Fu_conse_877 | ATTCCTTTGAGTT | Fusobacterium |
| | | | | TCATAC | consensus |
| 247 | 14 | 13 | Es_col_2 | CATGATCAAACT | Escherichiacol |
| | | | | CTCAATT | |
| 248 | 14 | 14 | En_cloac_120 | GGTCTTGCGACTT | Enterobacter cloacae |
| | | | | TATGCG | |
| 249 | 14 | 15 | Co_conse_956 | CATCGAATTAAT | Corynebacterium |
| | | | | CCACATG | consensus |
| 250 | 14 | 16 | Cl_conse_632 | TTTCACATCTGAC | Clostridium consensus |
| | | | | TTAAAT | |
| 251 | 14 | 17 | Ca_conse_228 | TCTCCAACTAGCT | Capnocytophaga |
| | | | | AATAGA | consensus |
| 252 | 14 | 18 | Ba_fragi_162 | CATCCTTTACCGG | Bacteroides fragilis |
| | | | | AATCCT | |
| 253 | 15 | 1 | CN_conse_243 | TTACCAACTAGC | CNS consensus |
| | | | | TAATACG | |
| 254 | 15 | 2 | Cl_bifer_88 | CCGTATTAGTAT | Clostridium |
| | | | | ACCTTTC | bifermentans |
| 255 | 15 | 3 | Bu_cepac_395 | CTGTATTAGAAC | Burkholderia cepacia |
| | | | | CAAGGAT | |
| 256 | 15 | 4 | Ba_fragi_448 | CTGCACTTTATTC | Bacteroides fragili |
| | | | | TTATAT | |
| 257 | 15 | 5 | Ae_virid_70 | GCTTATAGGTAG | Aerococcus viridans |
| | | | | ATTCCTT | |
| 258 | 15 | 6 | Ac_calco_397 | CTGAAGGTATTA | Acinetobacter |
| | | | | ACTTCAG | calcoaceticus |
| 259 | 15 | 7 | Sh_dysen_981 | GTATCTCTACAA | Shigella dysenteria |
| | | | | GGTTCTG | |
| 260 | 15 | 8 | St_aureu_446 | CACAGTTACTTA | Staphylococcus aureus |
| | | | | CACATAT | |
| 261 | 15 | 9 | Pr_penne_415 | TAACGTCAATTG | Proteus penneri |
| | | | | ATAAAGG | |
| 262 | 15 | 10 | Pe_micro_15 | AGAATTTCCACA | PeptoStreptococcus |
| | | | | AAAATCA | micros |
| 263 | 15 | 11 | Mo_osloe_409 | CAGGTAACGTCT | Moraxella osloensis |
| | | | | AATCTAA | |
| 264 | 15 | 12 | Kl_pneum_432 | ATCGATGAGGTT | Klebsiella pneumonia |
| | | | | ATTAACC | |
| 265 | 15 | 13 | Ge_morbi_148 | AACCAACTTTTA | Gemella morbillorum |
| | | | | AATATCT | |
| 266 | 15 | 14 | Fu_conse_1261 | CTAAGAATAGTT | Fusobacterium |
| | | | | TTCTGAG | consensus |
| 267 | 15 | 15 | En_faeci_1261 | TTTAAGAGATTA | Enterococcus faeciu |
| | | | | GCTTAGC | |
| 268 | 15 | 16 | En_agglo_1282 | GTAACATTCTGA | Enterobacter |
| | | | | TTTACGA | agglomeran |
| 269 | 15 | 17 | Co_conse_177 | CTAAAGTATGGT | Corynebacterium |
| | | | | GTCCTAT | consensus |
| 270 | 15 | 18 | Cl_clost_18 | GAAAACTTCATC | Clostridium |
| | | | | TTAATTG | clostridiiforme |
| 271 | 16 | 1 | En_faeca_436 | AGATACCGTCAG | Enterococcus faecalis |
| | | | | GGGACGT | |
| 272 | 16 | 2 | En_aggl._578 | AGAACTCAAGCT | Enterobacter aggl. |
| | | | | GCCAGTT | |
| 273 | 16 | 3 | CN_conse_1455 | TAGCTCCTAATA | CNS consensus |
| | | | | AATGGTT | |
| 274 | 16 | 4 | Ci_freun_1165 | CCTTCCTCTAGTT | Citrobacter freundi |
| | | | | TATCAC | |
| 275 | 16 | 5 | Bu_cepac_393 | GTATTAGAACCA | Burkholderia cepacia |
| | | | | AGGATTT | |
| 276 | 16 | 6 | Ba_fragi_453 | ACATACTGCACT | Bacteroides fragili |
| | | | | TTATTCT | |
| 277 | 16 | 7 | St_aureu_15 | AGCAGTTACTCT | Staphylococcus aureus |
| | | | | ACAATTT | |
| 278 | 16 | 8 | Ac_bauma_394 | TAGGTATTAACT | Acinetobacter |
| | | | | AAAGTAG | baumannii |
| 279 | 16 | 9 | Se_marce_464 | CAATTGATGAGC | Serratia marcescen |
| | | | | GTATTAA | |
| 280 | 16 | 10 | Ps_aerug_121 | ACTTTCTCCCTCA | Pseudomonas |
| | | | | GGACGT | aeruginosa |
| 281 | 16 | 11 | Pr_mirab_417 | GGTAACGTCAAT | Proteus mirabilis |
| | | | | TGATAAG | |
| 282 | 16 | 12 | Pe_micro_400 | CGTCATTATCTTC | PeptoStreptococcus |
| | | | | TCATAG | micros |
| 283 | 16 | 13 | Mo_catar_26 | ACTAAGTATCAG | Moraxella catarrhalis |
| | | | | AAGCAAG | |
| 284 | 16 | 14 | Kl_pneum_435 | TCAATCGATGAG | Klebsiella pneumonia |
| | | | | GTTATTA | |
| 285 | 16 | 15 | Ge_morbi_406 | TAGTTACTACAT | Gemella morbillorum |
| | | | | ATCCATT | |
| 286 | 16 | 16 | Fu_conse_1267 | TCCGAACTAAGA | Fusobacterium |
| | | | | ATAGTTT | consensus |
| 287 | 16 | 17 | En_faeca_89 | TTATCCCCCTCTG | Enterococcus faecalis |
| | | | | ATGGGT | |
| 288 | 16 | 18 | En_agglo_399 | GATGAAGTATTA | Enterobacter |
| | | | | ATTTCAC | agglomeran |

1. An analytical chip for the simultaneous determination of one or more different bacterial 16S-rRNA in a liquid sample
comprising
an evanescent field measurement platform, e.g. an optical waveguide, as a solid carrier and
a plurality of specific recognition elements immobilized in discrete measurement areas of known location forming an array of measurement areas on said evanescent field measurement platform,
wherein
a multitude (i.e. 2 or more) of different specific recognition elements is immobilized in discrete measurement areas for the recognition and detection of each different 16S-rRNA, different recognition elements being specific for different subsequences of the 16S-rRNA to be detected, which are not directly adjacent and not overlapping in the sequence of said 16S-rRNA, and
and said analytical chip is operable for the detection of 16S-rRNA in the evanescent field of the evanescent field measurement platform, without an amplification (e.g. by polymerase chain reaction PCR or linear amplification “T7”) of the polynucleotide sequences contained in the sample.

2. An analytical chip according to claim 1, wherein said analytical chip is operable for a simultaneous quantitative determination of one or more different bacterial 16S-rRNA in a liquid sample.

3. An analytical chip according to claim 1, wherein said analytical chip is operable for a simultaneous quantitative determination of the amount respectively concentration of the one or more different bacteria in the original sample from where the liquid sample containing said one or more different 16S-rRNA have been derived.

4. An analytical chip according to claim 1, wherein the one or more bacterial 16S-rRNA to be detected are derived from bacteria selected from the group comprising, e.g.:

| Genus | Species |
|---|---|
| Achromobacter | xylosoxidans |
| Acinetobacter | baumannii |
| Acinetobacter | calcoaceticus |
| Acinetobacter | junii |
| Acinetobacter | wolfii |
| Actinobacillus | sp |
| Actinomyces | israelii |
| Actinomyces | meyeri |
| Actinomyces | odontolyticus |
| Actinomyces | sp |
| Aerococcus | viridans |
| Aeromonas | caviae |
| Aeromonas | hydrophilia |
| Aeromonas | sobria |
| Agrobacterium | radiobacter |
| Alcaligenes | denitrificans |
| Alcaligenes | faecalis |
| Alcaligenes | sp |
| Alcaligenes | xylosoxydans |
| Bacillus | sp |
| Bacteroides | bivius |
| Bacteroides | buccae |
| Bacteroides | caccae |
| Bacteroides | denticola |
| Bacteroides | disiens |
| Bacteroides | distasonis |
| Bacteroides | fragilis |
| Bacteroides | oralis |
| Bacteroides | oris |
| Bacteroides | ovatus |
| Bacteroides | stercoris |
| Bacteroides | thetaiotomicron |
| Bacteroides | uniformis |
| Bacteroides | ureolyticus |
| Bacteroides | vulgatus |
| Bifidobacterium | sp |
| Bordetella | bronchiseptica |
| Brucella | melitensis |
| Burkholderia | cepacia |
| Burkholderia | picketti |
| Burkholderia | pseudomallei |
| Campylobacter | coli |
| Campylobacter | fetus |
| Campylobacter | jejuni |
| Campylobacter | sp |
| Capnocytophaga | canimorsus |
| Capnocytophaga | ochracea |
| Capnocytophaga | sp |
| Chryseomonas | luteola |
| Citrobacter | amalonaticus |
| Citrobacter | braakii |
| Citrobacter | diversus |
| Citrobacter | freundii |
| Citrobacter | koseri |
| Citrobacter | sp |
| Clostridium | bifermentans |
| Clostridium | butyricum |
| Clostridium | clostridiiforme |
| Clostridium | paraputrificum |
| Clostridium | perfringens |
| Clostridium | ramosum |
| Clostridium | septicum |
| Clostridium | tertium |
| Clostridiumi | innocuum |
| Comamonas | acidovora |
| Corynebacterium | aquaticum |
| Corynebacterium | bovis |
| Corynebacterium | jeikeium |
| Corynebacterium | minutissimum |
| Corynebacterium | sp |
| Eikenella | corrodens |
| Empedobacter | brevis |
| Entereococcus | casseliflavus |
| Enterobacter | aerogenes |
| Enterobacter | agglomerans |
| Enterobacter | amnigenus |
| Enterobacter | cloacae |
| Enterococcus | avium |
| Enterococcus | durans |
| Enterococcus | faecalis |
| Enterococcus | faecium |
| Enterococcus | gallinarium |
| Enterococcus | raffinosus |
| Escherichia | coli |
| Eubacterium | aerofaciens |
| Eubacterium | lentum |
| Eubacterium | limosum |
| Flavobacterium | breve |
| Flavobacterium | meningosepticum |
| Flavobacterium | sp |
| Fusobacterium | sp |
| Fusobacterium | mortiferum |
| Fusobacterium | necrophorum |
| Fusobacterium | nucleatum |
| Fusobacterium | varium |
| Gardnerella | vaginalis |
| Gemella | haemolysans |
| Gemella | morbillorum |
| Gemella | sp |
| Haemophilus | aphrophilus |
| Haemophilus | influenzae |
| Haemophilus | parainfluenzae |
| Haemophilus | paraphrophilus |
| Hafnia | alvei |
| Kingella | sp |
| Klebsiella | ornithinolytica |
| Klebsiella | oxytoca |
| Klebsiella | ozaenae |
| Klebsiella | pneumoniae |
| Kluyvera | sp |
| Lactobacillus | acidophilus |
| Lactobacillus | catenaforme |
| Lactococcus | cremoris |
| Lactococcus | lactis |
| Legionella | pneumophila |
| Leptotrichia | buccalis |
| Leuconostoc | sp |
| Listeria | monocytogenes |
| Moraxella | catarrhalis |
| Moraxella | osloensis |
| Moraxella | phenylpyruvica |
| Moraxella | sp |
| Morganella | morganii |
| Mycobacterium | avium |
| Mycobacterium | genavense |
| Mycobacterium | tuberculosis |
| Mycobacterium | avium-intracellulare |
| Mycoplasma | sp |
| Myroides | odoratum |
| Neisseria | cinerea |
| Neisseria | flavescens |
| Neisseria | meningitidis |
| Neisseria | mucosa |
| Neisseria | sp |
| Neisseria | subflava |
| Nocardia | asteroides |
| Nocardia | sp |
| Ochrobactrum | anthropi |
| Pasteurella | multocida |
| Peptostreptococcus | anaerobius |
| Peptostreptococcus | asaccharolyticus |
| Peptostreptococcus | magnus |
| Peptostreptococcus | micros |
| Peptostreptococcus | prevotii |
| Prevotella | bivia |
| Prevotella | buccae |
| Prevotella | loescheii |
| Propionibacterium | acnes |
| Propionibacterium | granulosum |
| Proteus | mirabilis |
| Proteus | penneri |
| Proteus | vulgaris |
| Providencia | rettgeri |
| Providencia | sp |
| Providencia | stuartii |
| Pseudomonas | aeroginosa |
| Pseudomonas | alcaligenes |
| Pseudomonas | diminuta |
| Pseudomonas | fluorescens |
| Pseudomonas | paucimobilis |
| Pseudomonas | putida |
| Pseudomonas | sp |
| Pseudomonas | stutzeri |
| Pseudomonas | vesicularis |
| Salmonella | enteritidis |
| Salmonella | paratyphi |
| Salmonella | typhi |
| Salmonella | typhimurium |
| Serratia | fonticola |
| Serratia | marcescens |
| Serratia | odorifera |
| Serratia | sp |
| Shigella | dysenteria |
| Shigella | flexneri |
| Shigella | sonnei |
| Sphingomonas | paucimobilis |
| Staphylococcus | aureus |
| Staphylococcus | auricularis |
| Staphylococcus | capitis |
| Staphylococcus | caprae |
| Staphylococcus | chromogenes |
| Staphylococcus | cohnii |
| Staphylococcus | epidermidis |
| Staphylococcus | haemolyticus |
| Staphylococcus | hominis |
| Staphylococcus | intermedius |
| Staphylococcus | kloosii |
| Staphylococcus | lugdunensis |
| Staphylococcus | saccharolyticus |
| Staphylococcus | saprophyticus |
| Staphylococcus | sciuri |
| Staphylococcus | simulans |
| Staphylococcus | warneri |
| Staphylococcus | xylosus |
| Stenotrophomonas | maltophilia |
| Stomatococcus | mucilaginosus |
| Streptococcus | acidiminimus |
| Streptococcus | adjacens |
| Streptococcus | agalactiae |
| Streptococcus | anginosus |
| Streptococcus | bovis |
| Streptococcus | canis |
| Streptococcus | constellatus |
| Streptococcus | cremoris |
| Streptococcus | crista |
| Streptococcus | defectivus |
| Streptococcus | dysgalactiae |
| Streptococcus | equinus |
| Streptococcus | equisimilis |
| Streptococcus | intermedius |
| Streptococcus | lactis |
| Streptococcus | mitis |
| Streptococcus | mutans |
| Streptococcus | oralis |
| Streptococcus | pneumoniae |
| Streptococcus | pyogenes |
| Streptococcus | salivarius |
| Streptococcus | sanguis |
| Streptococcus | alpha-hemolyticus |
| Streptococcus | beta-hemolyticus |
| Veillonella | parvula |
| Veillonella | sp |
| Yersinia | enterocolitica |

5. An analytical chip according to claim 1, wherein the immobilized specific recognition elements are selected from the group comprising, e.g., natural and synthetically fabricated polynucleotides, polynucleotides with artificial bases and/or artificial carbohydrates, peptides, peptide nucleic acids (“PNA”s), PNA's with artificial bases, LNAs, proteins (e.g. antibodies), ribozymes, and aptamers.

6. An analytical chip according to claim 1, wherein the immobilized specific recognition elements are selected from the group of antibiotics-based recognition elements comprising, e.g., macrolide antibiotics (e.g. erythromycin, azithromycin, streptogramin), aminoglycoside antibiotics (e.g. neomycin, paromomycin, lividomycin, gentamycin), and peptide antibiotics (e.g. thiostreptone, micrococcin).

7. An analytical chip according to claim 1, for the simultaneous determination of one or more different bacterial 16S-rRNA in a liquid sample,
comprising
an evanescent field measurement platform, e.g. an optical waveguide, as a solid carrier and
a plurality of polynucleotides immobilized in discrete measurement areas of known location forming an array of measurement areas on said evanescent field measurement platform,
wherein
a multitude (i.e. 2 or more) of different polynucleotides is immobilized in discrete measurement areas for the detection of each different 16S-rRNA, the sequences of the immobilized polynucleotides being essentially complementary to different subsequences of the 16S-rRNA to be detected, which are not directly adjacent and not overlapping in the sequence of said 16S-rRNA, and
and said analytical chip is operable for the detection of 16S-rRNA in the evanescent field of the evanescent field measurement platform, without an amplification (e.g. by polymerase chain reaction PCR or linear amplification “T7”) of the polynucleotide sequences contained in the sample.

8-10. (canceled)

11. An analytical chip according to claim 1, wherein the plurality of immobilized specific recognition elements comprises less than 10 different specific recognition elements which can bind specifically to different subsequences of the same bacterial 16S-rRNA to be detected.

12. An analytical chip according to claim 7, wherein the sequences of the multitude of immobilized polynucleotides for detection of a 16S-rRNA are essentially complementary to subsequences indicative for the genus of the bacterium from which said 16S-rRNA to be detected has been derived.

13. An analytical chip according to claim 7, wherein the sequences of the multitude of immobilized polynucleotides for detection of a 16S-rRNA are essentially complementary to subsequences indicative for the species and/or strain of the bacterium from which said 16S-rRNA to be detected has been derived.

14. An analytical chip according to claim 7, wherein the multitude of immobilized polynucleotides for detection of a 16S-rRNA comprises both polynucleotides with a sequence essentially complementary to subsequences indicative for the genus type and polynucleotides with a sequence essentially complementary to the species and/or strain of the bacterium from which said 16S-rRNA to be detected has been derived.

15. An analytical method according to claim 1, wherein the liquid sample comprises a complex biological matrix of the group of human and animal cell extracts, extracts of human and animal tissue, such as organ, skin or bone tissue, and of body fluids or their components, such as blood, serum, plasm, lymph, synovia, tear liquid, sweat, milk, sperm, sputum, cerebral spinal fluid, gastric juice, intestinal contents, urine, and stool.

16. (canceled)

17. An analytical chip according to claim 1, wherein the evanescent field measurement platform comprises an optical waveguide, which is continuous or partitioned into discrete waveguiding areas.

18. An analytical chip according to claim 17, wherein the optical waveguide is an optical film waveguide with a first optically transparent layer (a) on a second optically transparent layer (b) with lower refractive index than layer (a).

19-24. (canceled)

25. An analytical chip according to claim 18, wherein in-coupling of excitation light into the optically transparent layer (a), to the measurement areas, is performed using one or more grating structures (c), that are formed in the optically transparent layer (a).

26-29. (canceled)

30. An analytical chip according to claim 1, wherein an adhesion-promoting layer (f), with a thickness of preferably less than 200 nm, more preferably of less than 20 nm, is deposited on the optically transparent layer (a), for immobilization of the specific recognition elements, and wherein the adhesion-promoting layer preferably comprises chemical compounds of the group comprising, e.g., silanes, epoxides, functionalized, charged or polar polymers and “self-organized passive or functionalized mono- or multilayers”, alkyl phosphates or alkyl phosphonates, and multifunctional block copolymers, such as poly(L)lysine/polyethylene glycols.

31-34. (canceled)

35. An analytical chip according to claim 1, wherein the measurement areas are provided at a density of more than 10 measurement areas per square centimeter.

36. An analytical chip according to claim 1, wherein the surface with the discrete measurement areas with immobilized specific recognition elements forms the inner bottom surface of one or more sample compartments for receiving one or more samples to be analyzed for 16S-rRNA.

37-41. (canceled)

42. An analytical method for the simultaneous determination of one or more different bacterial 16S-rRNA in a liquid sample, comprising providing an analytical chip
comprising
an evanescent field measurement platform, e.g. an optical waveguide, as a solid carrier and
a plurality of specific recognition elements immobilized in discrete measurement areas of known location forming an array of measurement areas on said evanescent field measurement platform,
wherein
a multitude (i.e. 2 or more) of different specific recognition elements is immobilized in discrete measurement areas for the recognition and detection of each different 16S-rRNA, different recognition elements being specific for different subsequences of the 16S-rRNA to be detected, which are not directly adjacent and not overlapping in the sequence of said 16S-rRNA,
a liquid sample, not being subjected to an amplification (e.g. by polymerase chain reaction PCR or linear amplification “T7”) of the polynucleotide sequences contained therein, is brought into contact with the array under conditions allowing for binding (respectively hybridization) of 16S-rRNA contained in the sample with the corresponding specific recognition elements immobilized in the measurement areas
changes of electro-optical signal caused by a successful binding on the measurement areas of the evanescent field measurement platform are measured with one or more detectors, and
the presence of a bacterium to be detected is determined from the whole of signals from those measurement areas occupied by immobilized specific recognition elements dedicated for the specific detection of said bacterium.

43. An analytical method according to claim 42, wherein said analytical method is operable for a simultaneous quantitative determination of one or more different bacterial 16S-rRNA in a liquid sample.

44. An analytical method according to claim 42, wherein said analytical method is operable for a simultaneous quantitative determination of the amount respectively concentration of the one or more different bacteria in the original sample from where the liquid sample containing said one or more different 16S-rRNA have been derived.

45. An analytical method according to claim 42, wherein the one or more bacterial 16S-rRNA to be detected are derived from bacteria selected from the group comprising, e.g.:

| Genus | Species |
|---|---|
| Achromobacter | xylosoxidans |
| Acinetobacter | baumannii |
| Acinetobacter | calcoaceticus |
| Acinetobacter | junii |
| Acinetobacter | wolfii |
| Actinobacillus | sp |
| Actinomyces | israelii |
| Actinomyces | meyeri |
| Actinomyces | odontolyticus |
| Actinomyces | sp |
| Aerococcus | viridans |
| Aeromonas | caviae |
| Aeromonas | hydrophilia |
| Aeromonas | sobria |
| Agrobacterium | radiobacter |
| Alcaligenes | denitrificans |
| Alcaligenes | faecalis |
| Alcaligenes | sp |
| Alcaligenes | xylosoxydans |
| Bacillus | sp |
| Bacteroides | bivius |
| Bacteroides | buccae |
| Bacteroides | caccae |
| Bacteroides | denticola |
| Bacteroides | disiens |
| Bacteroides | distasonis |
| Bacteroides | fragilis |
| Bacteroides | oralis |
| Bacteroides | oris |
| Bacteroides | ovatus |
| Bacteroides | stercoris |
| Bacteroides | thetaiotomicron |
| Bacteroides | uniformis |
| Bacteroides | ureolyticus |
| Bacteroides | vulgatus |
| Bifidobacterium | sp |
| Bordetella | bronchiseptica |
| Brucella | melitensis |
| Burkholderia | cepacia |
| Burkholderia | picketti |
| Burkholderia | pseudomallei |
| Campylobacter | coli |
| Campylobacter | fetus |
| Campylobacter | jejuni |
| Campylobacter | sp |
| Capnocytophaga | canimorsus |
| Capnocytophaga | ochracea |
| Capnocytophaga | sp |
| Chryseomonas | luteola |
| Citrobacter | amalonaticus |
| Citrobacter | braakii |
| Citrobacter | diversus |
| Citrobacter | freundii |
| Citrobacter | koseri |
| Citrobacter | sp |
| Clostridium | bifermentans |
| Clostridium | butyricum |
| Clostridium | clostridiiforme |
| Clostridium | paraputrificum |
| Clostridium | perfringens |
| Clostridium | ramosum |
| Clostridium | septicum |
| Clostridium | tertium |
| Clostridiumi | innocuum |
| Comamonas | acidovora |
| Corynebacterium | aquaticum |
| Corynebacterium | bovis |
| Corynebacterium | jeikeium |
| Corynebacterium | minutissimum |
| Corynebacterium | sp |
| Eikenella | corrodens |
| Empedobacter | brevis |
| Entereococcus | casseliflavus |
| Enterobacter | aerogenes |
| Enterobacter | agglomerans |
| Enterobacter | amnigenus |
| Enterobacter | cloacae |
| Enterococcus | avium |
| Enterococcus | durans |
| Enterococcus | faecalis |
| Enterococcus | faecium |
| Enterococcus | gallinarium |
| Enterococcus | raffinosus |
| Escherichia | coli |
| Eubacterium | aerofaciens |
| Eubacterium | lentum |
| Eubacterium | limosum |
| Flavobacterium | breve |
| Flavobacterium | meningosepticum |
| Flavobacterium | sp |
| Fusobacterium | sp |
| Fusobacterium | mortiferum |
| Fusobacterium | necrophorum |
| Fusobacterium | nucleatum |
| Fusobacterium | varium |
| Gardnerella | vaginalis |
| Gemella | haemolysans |
| Gemella | morbillorum |
| Gemella | sp |
| Haemophilus | aphrophilus |
| Haemophilus | influenzae |
| Haemophilus | parainfluenzae |
| Haemophilus | paraphrophilus |
| Hafnia | alvei |
| Kingella | sp |
| Klebsiella | ornithinolytica |
| Klebsiella | oxytoca |
| Klebsiella | ozaenae |
| Klebsiella | pneumoniae |
| Kluyvera | sp |
| Lactobacillus | acidophilus |
| Lactobacillus | catenaforme |
| Lactococcus | cremoris |
| Lactococcus | lactis |
| Legionella | pneumophila |
| Leptotrichia | buccalis |
| Leuconostoc | sp |
| Listeria | monocytogenes |
| Moraxella | catarrhalis |
| Moraxella | osloensis |
| Moraxella | phenylpyruvica |
| Moraxella | sp |
| Morganella | morganii |
| Mycobacterium | avium |
| Mycobacterium | genavense |
| Mycobacterium | tuberculosis |
| Mycobacterium | avium-intracellulare |
| Mycoplasma | sp |
| Myroides | odoratum |
| Neisseria | cinerea |
| Neisseria | flavescens |
| Neisseria | meningitidis |
| Neisseria | mucosa |
| Neisseria | sp |
| Neisseria | subflava |
| Nocardia | asteroides |
| Nocardia | sp |
| Ochrobactrum | anthropi |
| Pasteurella | multocida |
| Peptostreptococcus | anaerobius |
| Peptostreptococcus | asaccharolyticus |
| Peptostreptococcus | magnus |
| Peptostreptococcus | micros |
| Peptostreptococcus | prevotii |
| Prevotella | bivia |
| Prevotella | buccae |
| Prevotella | loescheii |
| Propionibacterium | acnes |
| Propionibacterium | granulosum |
| Proteus | mirabilis |
| Proteus | penneri |
| Proteus | vulgaris |
| Providencia | rettgeri |
| Providencia | sp |
| Providencia | stuartii |
| Pseudomonas | aeroginosa |
| Pseudomonas | alcaligenes |
| Pseudomonas | diminuta |
| Pseudomonas | fluorescens |
| Pseudomonas | paucimobilis |
| Pseudomonas | putida |
| Pseudomonas | sp |
| Pseudomonas | stutzeri |
| Pseudomonas | vesicularis |
| Salmonella | enteritidis |
| Salmonella | paratyphi |
| Salmonella | typhi |
| Salmonella | typhimurium |
| Serratia | fonticola |
| Serratia | marcescens |
| Serratia | odorifera |
| Serratia | sp |
| Shigella | dysenteria |
| Shigella | flexneri |
| Shigella | sonnei |
| Sphingomonas | paucimobilis |
| Staphylococcus | aureus |
| Staphylococcus | auricularis |
| Staphylococcus | capitis |
| Staphylococcus | caprae |
| Staphylococcus | chromogenes |
| Staphylococcus | cohnii |
| Staphylococcus | epidermidis |
| Staphylococcus | haemolyticus |
| Staphylococcus | hominis |
| Staphylococcus | intermedius |
| Staphylococcus | kloosii |
| Staphylococcus | lugdunensis |
| Staphylococcus | saccharolyticus |
| Staphylococcus | saprophyticus |
| Staphylococcus | sciuri |
| Staphylococcus | simulans |
| Staphylococcus | warneri |
| Staphylococcus | xylosus |
| Stenotrophomonas | maltophilia |
| Stomatococcus | mucilaginosus |
| Streptococcus | acidiminimus |
| Streptococcus | adjacens |
| Streptococcus | agalactiae |
| Streptococcus | anginosus |
| Streptococcus | bovis |
| Streptococcus | canis |
| Streptococcus | constellatus |
| Streptococcus | cremoris |
| Streptococcus | crista |
| Streptococcus | defectivus |
| Streptococcus | dysgalactiae |
| Streptococcus | equinus |
| Streptococcus | equisimilis |
| Streptococcus | intermedius |
| Streptococcus | lactis |
| Streptococcus | mitis |
| Streptococcus | mutans |
| Streptococcus | oralis |
| Streptococcus | pneumoniae |
| Streptococcus | pyogenes |
| Streptococcus | salivarius |
| Streptococcus | sanguis |
| Streptococcus | alpha-hemolyticus |
| Streptococcus | beta-hemolyticus |
| Veillonella | parvula |
| Veillonella | sp |
| Yersinia | enterocolitica |

46. An analytical method according to claim 42, wherein the immobilized specific recognition elements are selected from the group comprising, e.g., natural and synthetically fabricated polynucleotides, polynucleotides with artificial bases and/or artificial carbohydrates, peptides, peptide nucleic acids (“PNA”s), PNA's with artificial bases, LNAs, proteins (e.g. antibodies), ribozymes, and aptamers.

47. An analytical method according to claim 42, wherein the immobilized specific recognition elements are selected from the group of antibiotics-based recognition elements comprising, e.g., macrolide antibiotics (e.g. erythromycin, azithromycin, streptogramin), aminoglycoside antibiotics (e.g. neomycin, paromomycin, lividomycin, gentamycin), and peptide antibiotics (e.g. thiostreptone, micrococcin).

48. An analytical method according to claim 42, for the simultaneous determination of one or more different bacterial 16S-rRNA in a liquid sample, comprising providing an analytical chip comprising
an evanescent field measurement platform, e.g. an optical waveguide, as a solid carrier and
a plurality of polynucleotides immobilized in discrete measurement areas of known location forming an array of measurement areas on said evanescent field measurement platform,
wherein
a multitude (i.e. 2 or more) of different polynucleotides is immobilized in discrete measurement areas for the detection of each different 16S-rRNA, the sequences of the immobilized polynucleotides being essentially complementary to different subsequences of the 16S-rRNA to be detected, which are not directly adjacent and not overlapping in the sequence of said 16S-rRNA,
a liquid sample, not being subjected to an amplification (e.g. by polymerase chain reaction PCR or linear amplification “T7”) of the polynucleotide sequences contained therein, is brought into contact with the array under conditions allowing a hybridization of 16S-rRNA contained in the sample with essentially complementary polynucleotides immobilized in the measurement areas
changes of electro-optical signal caused by a successful hybridization on the measurement areas of the evanescent field measurement platform are measured with one or more detectors, and
the presence of a bacterium to be detected is determined from the whole of signals from those measurement areas occupied by immobilized polynucleotides dedicated for the specific detection of said bacterium.

49-51. (canceled)

52. An analytical method according to claim 42, wherein the plurality of immobilized specific recognition elements comprises less than 10 different specific recognition elements which can bind specifically to different subsequences of the same bacterial 16S-rRNA to be detected.

53. (canceled)

54. An analytical method according to claim 42, wherein the bacterial 16S-rRNA to be detected is fragmented into strands of less than 500, preferably of less than 200 base pairs length.

55. (canceled)

56. An analytical method according to claim 42, wherein the evanescent field measurement platform comprises an optical waveguide, which is continuous or partitioned into discrete waveguiding areas.

57. An analytical method according to claim 56, wherein the optical waveguide is an optical film waveguide with a first optically transparent layer (a) on a second optically transparent layer (b) with lower refractive index than layer (a).

58. An analytical method according to claim 42, wherein the detection of the presence of bacterial 16S-rRNA is based on the change of one or more luminescences, preferably of one or more fluorescences.

59. An analytical method according to claim 58, wherein the luminescence (fluorescence) used for analyte detection is generated by luminescence (fluorescence) labels, which are bound to or associated with the 16S-rRNA to be detected.

60. (canceled)

61. An analytical method according to claim 59, wherein said labels have excitation and emission wavelengths between 250 nm and 1100 nm.

62. An analytical method according to claim 59, wherein said luminescence labels are selected from luminescent, functionalized or intercalating dyes and luminescent, functionalized nanoparticles (“quantum dots”).

63. (canceled)

64. An analytical method according to claim 48, wherein a pattern of said changes of electro-optical signal caused by a successful hybridization of a multitude of immobilized polynucleotides, in different measurement areas, dedicated for the detection of one or more 16S-rRNA, (“sample hybridization pattern” of said 16S-rRNA) to be determined in a sample is established and recorded.

65. An analytical method according to claim 48, wherein a “reference hybridization pattern” is established and recorded by bringing a liquid sample containing a known amount of one or more different known 16S-rRNA into contact with said analytical chip under conditions allowing for hybridization between said known 16S-rRNA and the corresponding multitudes of complementary immobilized polynucleotides.

66. (canceled)

67. An analytical method according to claim 65, wherein 16S-rRNA contained in a sample are determined by comparison of a sample hybridization pattern and one or more reference hybridization patterns, upon determining the degree of agreement between said sample hybridization pattern and said reference hybridization patterns.

68. An analytical method according to claim 67, wherein the degree of agreement between said sample hybridization pattern and said reference hybridization patterns is determined by statistical methods and/or mathematical clustering methods and/or artificial neural networks.

69-70. (canceled)

71. An analytical method according to claim 42, wherein a pattern of said changes of electro-optical signal caused by a successful binding of a multitude of immobilized specific recognition elements in different measurement areas, dedicated for the detection of one or more 16S-rRNA, (“sample binding pattern” of said 16S-rRNA) to be determined in a sample is established and recorded.

72. An analytical method according to claim 42, wherein a “reference binding pattern” is established and recorded by bringing a liquid sample containing a known amount of one or more different known 16S-rRNA into contact with said analytical chip under conditions allowing for binding between said known 16S-rRNA and the corresponding multitudes of complementary immobilized specific recognition elements.

73. (canceled)

74. An analytical method according to claim 72, wherein 16S-rRNA contained in a sample are determined by comparison of a sample binding pattern and one or more reference binding patterns, upon determining the degree of agreement between said sample binding pattern and said reference binding patterns by statistical methods and/or mathematical clustering methods and/or artificial neural networks.

75-77. (canceled)
